(19) United States
(12) Patent Application Publication
(10) Pub. No.: US 2007/0112187 A1
(43) Pub. Date: May 17, 2007

(54) Separation process

(76) Inventors: Heikki Heikkila, Espoo (FI); Hannu Koivikko, Kantvik (FI); Juha Nurmi, Kirkkonummi (FI); Jari Mattila, Kirkkonummi (FI); Pia Saari, Helsinki (FI); Nina Nurmi, Helsinki (FI); Paivi Sarmala, Rajamaki (FI); Mirja Lindroos, Kirkkonummi (FI); Jari Lewandowski, Siuntio (FI)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/587,870

(22) PCT Filed: Oct. 29, 2004
(86) PCT No.: PCT/FI04/00641

§ 371 Date: Dec. 18, 2006

Related U.S. Application Data
(63) Continuation-in-part of application No. 10/697,763, filed on Oct. 30, 2003, now abandoned.

(51) Int. Cl. C07H 1/00

(52) U.S. Cl. 536/124

(57) ABSTRACT

The invention relates to a process of recovering arabinose and optionally other monosaccharides from vegetable fiber rich in heteropolymeric arabinose, such as gum arabic. Said other monosaccharides are typically selected from galactose and rhamnose. The process of the invention comprises controlled hydrolysis of the arabinose-rich vegetable fiber and fractionation of the hydrolysis product to obtain a fraction enriched in arabinose and optionally other product fractions followed by crystallization of arabinose. The invention also relates to a novel method of crystallizing arabinose from biomass-derived material. Furthermore, the invention relates to novel crystalline L-arabinose.

### FIELD OF THE INVENTION

The invention relates to the field of sugar separation technology and more particularly to a process of recovering arabinose and optionally at least one other monosaccharide typically selected from galactose, rhamnose and mannose from vegetable fiber which is rich in heteropolymeric arabinose and further contains other monosaccharides. The invention also relates to crystal line L-arabinose obtained by the process. Furthermore, the invention relates to a novel process of crystallizing arabinose from biomass-derived material. The invention also relates to the use of the crystalline arabinose product in pharmaceuticals and foodstuffs.

### BACKGROUND OF THE INVENTION

L-arabinose is found in the cell walls and pectic compounds of practically all green plants. However, as a rule, arabinose does not occur in the form of a free sugar, but as a constituent of complex heteropolysaccharides further containing galactose, galacturonic acid, glucuronic acid, 4-O-methyl-glucuronic acid, xylose, rhamnose and ferulic acid, for example. To recover arabinose from plant-based materials, the arabinose-containing polysaccharides must first be hydrolyzed to release arabinose to the form of a free sugar. Arabinose is then recovered from the hydrolyzate by various methods.

In accordance with Merck Index, 12^{th} edition, 1996, crystalline L-arabinose has a melting point of 157 to 160° C.

Arabinose is used in the pharmaceutical industry, for example as a pharmaceutical excipient or intermediate. Arabinose has also applications in food technology, for instance as a flavour ingredient or a non-caloric sweetener.

L-arabinose is recommended as a substitute for D-glucose in the diet for diabetic patients [Drzhevetskaya, A., Byul. Eksperim. Biol. Med. 61 (1966) 40; Chem. Abstr. 65 (1966) 9482 d].

Arabinose has been found useful in preventing or treating hyperglycemia (EP 0 560 284, Kodo Shusei K K, published 15 Sep. 1993). Said reference discloses a preparation and a method for preventing or treating hyperglycemia, whereby the preparation comprises, as an active ingredient, at least one component selected from the group consisting of L-arabinose, L-fucose, 2-deoxy-D-galactose, D-xylose, D-ribose, D-tagatose, D-ribulose, D-lyxose and D-xylulose. Furthermore, it is recited in Japanscan Food Industry Bulletin Oct. 8, 1994, p. 16 (Abstract) that an addition of 1 to 2% L-arabinose brings down elevated blood sugar levels and decreases high calorie intake by inhibiting sucrase.

JP 2002153245 (Asahi Soft Drinks Co., published 28 May, 2002) also discloses that arabinose suppresses the ingestion of calories in the human body, whereby it is especially useful in diet foodstuffs, such as diet drinks. JP 2002136278 (Unitika Ltd, published 14 Apr. 2002) discloses that arabinose has a controlling effect on the blood glucose level. Said reference describes an arabinose-containing fruit or vegetable juice which contains an L-arabinose-containing fraction obtained from enzymatic treatment of strained lees of fruits or vegetables containing arabinan, arabinoxylan or arabinogalactan.

EP 1 340 504, Fujii Makoto et al. (published 3 Sep. 2003) discloses a remedy for diabetes mellitus, containing L-arabinose and sucrose as active ingredients. It is recited that the source of sucrose in said remedy may be sugar or a sugar-containing food or beverage.

L-arabinose is also recited to have a boosting effect on the immune system, like Ginseng and Echinacea (Natura Internacional S. L., www.ricote.biz/sugars/).

Prior art teaches to recover arabinose from plant-based material, such as arabic gum, by extraction with an alkali, by hydrolysis with an acid or enzymes, followed by separation through precipitation of the polymeric material, or separation with chromatographic methods using organic eluents, ion exchange methods and/or through fermentation for removing hexoses, for example. The known processes are complex multistep processes and/or involve the use of organic solvents. Furthermore, the known processes do not as a rule provide arabinose which has a sufficient degree of purity for pharmaceutical or food applications, for example.

U.S. Pat. No. 4,772,334, Kureha Kagaku Kogyo Kabushiki Kaisha (published Sep. 20, 1988) discloses a process for producing highly pure rhamnose from gum arabic. The process comprises (a) partial hydrolysis of gum arabic in an aqueous solution of mineral acid to the extent that ⅓ to ½ of the constructing saccharides of the gum arabic is converted into L-rhamnose, L-arabinose and D-galactose to produce a liquid hydrolyzate comprising said monosaccharides, (b) neutralizing the liquid hydrolyzate with an alkali to a pH of 6.5 to 7.7 to obtain a neutralized hydrolyzate, (c) condensing the neutralized hydrolyzate by evaporation to obtain a solution containing 40 to 70% by weight of said monosaccharides, (d) adding a polar organic solvent to precipitate an insoluble substance, (e) removing the insoluble substance by centrifugation, filtration or sedimentation, (f) removing the polar organic solvent by evaporation to produce a solvent-free aqueous solution, (g) subjecting said solvent-free aqueous solution to strongly cationic ion-exchange resin chromatography to remove mainly D-galactose and L-arabinose using a mixture of water and acetone/acetonitrile as an eluent to produce a chromatographically purified aqueous solution, and (h) treating the rhamnose-containing aqueous solution with activated carbon to remove colored substances. Said polar organic solvent is typically selected from ethanol, isopropyl alcohol, acetone or acetonitrile. Said strongly acid cation exchange resin is typically used in Na⁺ form. This process is a complex multistep process. Furthermore, the process has the disadvantage that organic solvents are involved. Arabinose and galactose are not recovered.

CN 1,373,135 A, Univ. Tianjin (published 9 Oct. 2002) discloses a process for recovering L-arabinose from acacia gum. As a first step, the process comprises hydrolysis in an inorganic acid (including sulphuric acid), neutralization with an alkali, extraction with an alcohol, filtration and dissolving in acetic acid at 60 to 90° C. to obtain a crude L-arabinose mixture. The hydrolysis is followed by separation in a first chromatographic column to obtain a mixture of pure rhamnose and a mixture of L-arabinose and galactose, and separation in a second chromatographic column to obtain pure L-arabinose. The column filling materials for the chromatographic separation are selected from cellulose, alumina, starch and silica gel. The eluent for the separation is a mixture of water and organic solvents (n-butanol, ethyl acetate, isopropanol and acetic acid). In Examples 1 and 2, an arabinose product having a purity of 96% and 99.5% is obtained. The process is a multistep process involving the use of organic solvents.

A. Agarwal & P. L. Soni (Indian Journal of Chemistry, Section B, Organic Chemistry Including Medicinal Chemistry, 1988) discloses structural investigations of an acacia-catechu khair gum polysaccharide. It is recited that a purified exudate gum from khair (Acacia catechu) contains D-galactose, L-arabinose, L-rhamnose and D-glucuronic acid in a molar ratio of approximately (14.4):(5.4):(1.5):1. The degraded gum polysaccharides have been prepared by autohydrolysis in an aqueous solution of 0.001 N sulphuric acid at 100° C.

M. E. Osman et al., Phytochemistry (Oxford), vol. 38 (1995), No. 2, pp. 409 to 417 discloses the characterization of gum arabic fractions obtained by anion-exchange chromatography. Samples of gum arabic were fractionated by anion-exchange chromatography on DEAE-cellulose. It is recited that the carbohydrate compositions of all fractions were relatively constant with each containing similar proportions of galactose, arabinose, rhamnose and glucuronic acid.

G. O. Aspinall et. al., Journal of the Chemical Society, Abstracts (1958) 4408-14, discloses analytical studies on neutral oligosaccharides formed on partial acid hydrolysis of gum ghatti. The process is a complex multistep process comprising several hydrolysis steps with sulphuric acid (partial and complete hydrolyzations), neutralizations, treatments with Amberlite IR-120 (in H⁺ form) and IR-4B (in OH⁻ form), C-celite and cellulose. It is recited that monosaccharides such as arabinose, galactose, xylose and rhamnose were present in the fractions obtained from the treatment with Amberlite IR-120 and IR-4B, C-celite and cellulose.

G. O. Aspinall & J. Baillie, Journal of Chemical Society, Abstracts (1963) 1714-21, discloses the hydrolysis of methylated derivatives of arabinogalactan from gum tragacanth and an analysis of the hydrolysis products. It is disclosed that the hydrolysis provides 2,3,5-tri-, 2,3-, 2,5- and 3,5-di-, 2- and 3-O-methyl-L-arabinose, L-arabinose, 2,4,6-tri-, 2,3- and 2,4-di- and 2-O-methyl-D-galactose, D-galactose, 4-O-methyl-L-rhamnose, 2,3,4-tri- and 2,3-di-O-methyl-D-galacturonic acid and traces of other sugars. It is also recited that degraded polysaccharides were prepared by mild acid hydrolysis and by degradation of the periodate-oxidized arabinogalactan. The cleavage products were examined by chromatographic techniques.

The results of the studies of G. O. Aspinall et al. and M. E. Osman et al. above show the heteropolymeric structure of gum arabic, gum ghatti and gum tragacanth, which makes the recovery of pure arabinose difficult from vegetable fiber.

T. R. Ingle et al., Research and Industry (1985), 30(4), 369 to 673 discloses processes for the production of L-arabinose from gum ghatti (batches of 1.5 kg and 100 kg) by hydrolysis in diluted H₂SO₄, precipitation with an alcohol, neutralization with barium hydroxide, repeated treatments with an alcohol, crystallization from an alcohol and purification. It is recited that the process provides pure crystalline arabinose having a melting point of 159 to 160° C.

Ho Park Nyun et. al., Biotechnology Letters, 23(5), 411 to 416 (March 2001), discloses a method for the preparation of crystalline L-arabinose from arabinoxylan by enzymatic hydrolysis and selective fermentation with a yeast. Arabinoxylan corn fiber, which contained 28.1% (w/w) L-arabinose and 32.8% (w/w) D-xylose, was hydrolyzed with a crude enzyme containing β-xylanase, β-xylosidase and α-L-arabinofuranosidase originating from the extracellular culture broth of Penicillium funiculosum. The resultant hydrolysate contained L-arabinose, D-xylose and small amounts of other mono- and oligosaccharides. The hydrolysate was subjected to aerobic cultivation with Williopsis saturnus var saturnus, which metabolizes D-xylose without using L-arabinose. After the removal of yeast cells, the solution was decolorized with activated carbon and deionized with cation and anion exchange resins. The crystallization of L-arabinose from the solution provided crude L-arabinose crystals with a yield of 16% (based on the initial arabinoxylan).

SU 1009470 A, As. Kirg. Org. Chem. (published 17 Apr. 1983) discloses the preparation of arabinose from the gum of Rosaceae tree. The preparation process comprises treatment of the gum of Rosaceae tree with dilute sulphuric acid, neutralization, treatment of the neutralized material with boiling isopropanol, evaporation to a dry solids content of 40 to 46% and crystallization from isopropanol (70 to 75%).

EP 1 076 100, Sanwa Kosan K K (published 14 Feb. 2001) discloses a process of producing L-arabinose by contacting vegetable fibers with an acid to hydrolyze the fibers under conditions where L-arabinose contained in the vegetable fibers is selectively produced. The vegetable fiber used as the starting material typically contains 10% or more of L-arabinose as part of the constituting saccharides on the basis of the dry substance of the vegetable fiber. It is proposed that the starting material may be selected from corn husks, wheat bran, barley bran, oat bran, rye bran, rice bran, sugar beet fiber and apple fiber. In the examples, corn grain hulls were used as the starting material. The hydrolysis is typically carried out under conditions where the concentration of the acid is 0.01N to 0.50N, the dry substance concentration of the vegetable fiber being 3 to 20% by weight and the temperature being 80 to 150° C. In one embodiment of the process, the total amount of the saccharides decomposed and extracted during the hydrolysis is 30% or more (on the basis of the dry substance to be hydrolyzed) and the amount of L-arabinose in the total amount of the acid-hydrolyzed monosaccharides is 50% or more.

DD 143 261, Akademie derWissenschaften der DDR (published 13 Aug. 1980) discloses a process of recovering L-(+)-arabinose from sugar beet material by extraction with water, extraction with Ca(OH)₂ and hydrolysis with H₂SO₄, followed by crystallization. It is recited that the process provides crystalline L-arabinose, which has a melting point of 145 to 150° C.

CS 153 378, F. Janacek et al. (published May 15, 1974) discloses the production of L-arabinose and pectin from beet pulp by hydrolysis at 95° C. in 1% H₂SO₄, filtration, partial evaporation of the filtrate in vacuum, precipitation of pectin with EtOH, neutralization of the EtOH filtrate with Ca(OH)₂, fermentation with a yeast to remove D-galactose and sucrose, taking up the L-arabinose in 96% EtOH, purification by ion-exchange treatment and crystallization of the L-arabinose.

CS 137 537, V. Tibensky et al. (published 15 Jul. 1970) discloses hydrolysis of araban of sugar beet pulp by 50% H₂SO₄, followed by neutralization with lime or NH₃. Glucose and fructose are fermented with a yeast, the solution is evaporated to about 65% by weight, EtOH or MeOH is added, the solution is filtered and L-arabinose is crystallized from the alcohol-containing filtrate.

CS 139 427, V. Tibensky et al. (published 15 Dec. 1970) discloses the recovery of L-arabinose by hydrolysis of the L-arabinan of sugar beet with crude commercial arabinanase enzymes after conversion of protopectin to Ca-pectate. After filtration, arabinose may be purified from the solution by precipitation with alcohol or ion-exchange treatment.

CS 181 485, A. Kramar et al. (published 15 Jan. 1980) discloses studies on the recovery of pentoses, such as L-arabinose and xylose from beech bark by treatment with varying concentrations of H₂SO₄ (1% and 2% H₂SO₄) and varying temperatures (100° C. and 120° C.).

U.S. Pat. No. 6,506,897 B1, Danisco Finland (published Jan. 14, 2003) discloses a method of preparing crystalline L-arabinose from sugar beet pulp. The method comprises (a) extraction of sugar beet pulp, from which sugar has been extracted, in a strong alkaline solution, (b) hydrolysis of the crude araban thus obtained with a strong acid at an elevated temperature, (c) neutralization and filtration of the solution thus obtained, (d) chromatographic separation of the L-arabinose fraction by using a cation exchanger in a monovalent metal form as the separation resin, (e) purification of the L-arabinose solution thus obtained by means of cation and anion exchangers and adsorbent resins, and (f) recovery of pure L-arabinose as a crystalline product. The monovalent cation exchanger in step (d) is typically in Na⁺ form. In accordance with Example 1, the crystallization is carried out by cooling crystallization resulting in arabinose crystals, which have a purity over 98%. The process involves extraction in a strong alkaline medium as a pre-treatment step.

WO 01/21271 A1, Sohkar Oy (published 29 Mar. 2001) discloses a method for the chromatographic fractionation of pectin-containing vegetable material in the form of a pectin-containing aqueous solution with a cation exchange resin to provide a pectin fraction and optionally a salt fraction as well as a sugar fraction or fractions. Said sugar fraction may be an arabinose fraction, for example. The cation exchange resin is typically in Ca²⁺ or Al³⁺ form. The vegetable material is typically obtained from sugar beet pulp, citrus fruit or apples.

WO 01/21272 A1, Sohkar Oy (published 29 Mar. 2001) discloses a method of simultaneous purification and separation of pectin and pectic sugars/oligomers from sugar beet pulp by a multi-step process in an aqueous solution comprising hydrolysis, solids separation, fractionation by chromatography or ultrafiltration and recovering pectin and pectic sugars/oligomers. Pectic sugars typically comprise arabinose, for example. Examples 1 and 5 disclose a chromatographic separation process, which results in a sugar fraction containing about 85% L-arabinose. It is also recited that L-arabinose may be recovered from the sugar fraction by crystallization.

U.S. Pat. No. 4,816,078, Süddeutsche Zucker-Aktiengesellschaft (published 18 Mar. 1989) discloses a process for the production of crystalline L-arabinose from an araban-containing plant material, such as extracted sugar beet pulp. The process comprises (a) dissolving the araban in the presense of Ca(OH)₂ at a temperature of 105° C. to 160° C. in a closed vessel for a reaction period of 2 to 20 minutes, (b) neutralization of the resulting solution with an acid, followed by filtration, (c) concentrating the aqueous phase thus obtained to 40 to 60% by weight of araban by evaporation, followed by separation with a strong acid, weakly cross-linked cationic exchanger in Ca²⁺ form to obtain an araban-containing fraction and a by-product fraction, (d) hydrolyzing the araban-containing fraction with H₂SO₄, (e) neutralizing the hydrolyzed solution by adding CaCO₃ and concentrating the solution thus obtained to 40 to 60% by evaporation, (f) separating the concentrated solution with the same resin as in step (c) to obtain an L-arabinose-containing fraction and a by-product fraction, and (g) subjecting the arabinose-containing fraction to crystallization to obtain crystalline L-arabinose. In accordance with Example 1, the crystallization is carried out by multi-step cooling crystallization, which results in an arabinose purity of 95%. The process is a complex multistep process, where the separation of arabinose is preceded by the separation of the araban.

U.S. Pat. No. 4,516,566, Union Carbide Corporation (published May 14, 1985) discloses a process for the separation of arabinose from a liquid mixture containing arabinose and at least one other aldose sugar. The process comprises contacting said liquid mixture with an adsorbent comprising a BaX crystalline aluminosilicate zeolite to adsorb arabinose, followed by desorbing arabinose from said adsorbent with a desorbing agent, which is typically water. It is recited that said liquid mixture may be derived from the hydrolysis of wood. In addition to arabinose, said liquid mixture typically contains galactose, sucrose, glucose, fructose, mannose, xylose and cellobiose. The process involves the separation of arabinose from disaccharides (sucrose and cellobiose).

U. Kröplien [Carbohydrate Research, 32 (1974), pp. 167 to 170] has studied the interactions of aqueous solutions of sugars (including L-rhamnose, D-xylose, L-arabinose and D-galactose) with alumina. It is recited that by proper choise of alumina, sugars can be easily and quickly separated, both on a preparative as well as an analytical scale.

U.S. Pat. No. 4,880,919, UOP (published Nov. 14, 1989) discloses a process for fractionating an aqueous feed mixture containing arabinose and at least one other monosaccharide selected from aldopentoses and aldohexoses by contacting said mixture with an adsorbent which comprises a cation exchange resin including Ca²⁺ and NH₄⁺ ions to adsorb arabinose and then desorbing arabinose from said adsorbent with a desorbing agent. In addition to arabinose, said feed mixture may contain xylose, glucose, galactose, mannose and rhamnose.

WO 02/27039 A1, Xyrofin Oy (published 4 Apr. 2002) discloses a method of recovering a monosaccharide selected from the group consisting of rhamnose, arabinose, xylose and mixtures thereof from a solution containing at least two of said monosaccharides. The method is a multistep process comprising at least one step where a weakly acid cation exchange resin is used for the chromatographic separation. The ion form of said weakly acid cation exchange resin is typically selected from Na⁺, Mg²⁺, H⁺ and Ca²⁺. The process may also comprise a step where a strongly acid cation exchange resin is used as the separation resin. The starting solution is typically a hydrolyzate or a prehydrolyzate of hemicellulose from hardwood or xylose-containing biomass, which are not rich in arabinose. Example 9 discloses the crystallization of arabinose by boiling and cooling crystallization. The purity of the crystalline arabinose product is not disclosed.

Finnish Patent Application 20012605, Danisco Sweeteners Oy (published 1 Jul. 2003) discloses a method of recovering mannose from a solution derived from biomass by subjecting said solution to a chromatographic separation process using at least one chromatographic separation resin which is at least partly in Ba²⁺ form and at least one chromatographic separation resin which is in a form other than Ba²⁺ form. The latter resin is a cation exchange resin, where the cation is preferably Ca²⁺. The process may also comprise separation of arabinose. The starting biomass-derived solution is typically a hardwood spent liquor containing mannose in admixture with other sugars, such as xylose, galactose, glucose, rhamnose, arabinose and fructose. The starting material is not rich in arabinose.

U.S. Pat. No. 6,548,662 B1, Sanwa Kosan K K (published Apr. 15, 2003) discloses a method of fractionating a saccharide solution, where a feedstock solution obtained from hydrolysis of plant tissues and containing arabinose and an oligosaccharide where arabinose and/or xylose is/are the constituting component(s) is subjected to chromatographic fractionation in a simulated moving bed system. A concentrated L-arabinose solution and concentrated oligosaccharide solution are extracted from the system. Furthermore, a D-xylose fraction may be extracted from the system. A strongly acid cation-exchange resin in an alkaline earth metal form, preferably in a calcium salt form is typically used as the adsorbent for the chromatographic system. The plant hydrolyzate used as the starting material is typically derived from those containing large amounts of L-arabinose, such as corn husks, wheat bran, rice bran and squeezed lees of sugar beet or apple. The hydrolysis of the plant tissue is preferably carried out with a diluted acid (0.01 to 0.5N) at 80 to 150° C.

U.S. Pat. No. 6,262,318 B1, Xyrofin Oy (published Jul. 17, 2001) discloses a method of producing xylitol and erythritol from arabinoxylan-containing material, preferably corn and barley fibers. The method comprises hydrolyzing the arabinoxylan-containing material to obtain a hydrolyzate, separating xylose and arabinose from said hydrolyzate, reducing xylose to xylitol, recovering said xylitol, subjecting said arabinose to alkaline oxidation to obtain erythronic acid, reducing said erythronic acid to erythritol and recovering said erythritol. Said separation of arabinose and xylose is preferably carried out by chromatographic methods, typically by a two-step process comprising separation with a resin in Na⁺ form and separation with a resin in Ca²⁺ form. It is also recited that xylose and arabinose may be recovered by crystallization.

Y. Takasaki has studied the separation of sugars (including arabinose and galactose) on an anion-exchange resin in the bilsulphite form in Agr. Biol. Chem., Vol. 36, No. 13, p. 2575 to 2577, 1972.

It appears from the above-described background art that arabinose-rich raw materials are very complex multicomponent mixtures. Furthermore, arabinose-rich raw materials as a rule also contain relatively high amounts of galactose. Very complicated multistep processes involving the use of organic solvents have thus been required in the prior art to separate and crystallize arabinose from arabinose-rich sources which also contain galactose. On the other hand, the processes may require additional pre-treatment steps to extract and concentrate the arabinose-containing constituents. Many of the known processes for the recovery of arabinose are thus complicated and time-consuming for practical purposes. As a further disadvantage, the purity of the arabinose product has not always been sufficient for pharmaceutical and food applications, for example.

### BRIEF DESCRIPTION OF THE INVENTION

An object of the present invention is to provide a process of recovering arabinose and optionally at least one other monosaccharide selected from galactose, rhamnose and mannose from arabinose-rich sources so as to alleviate the disadvantages relating to the known processes described above. The objects of the invention are achieved by a process which is characterized by what is stated in the independent claims. Preferred embodiments of the invention are disclosed in the dependent claims.

The invention is based on a combination of controlled hydrolysis, fractionation by chromatography or membrane filtration and crystallization to recover arabinose from vegetable fiber rich in heteropolymeric arabinose.

It has been found in accordance with the present invention that arabinose can be separated and crystallized with high purity from arabinose-rich sources without significant disturbing effects of galactose. The whole process for the recovery of arabinose and optionally other monosaccharides and further products may preferably be carried out in an aqueous solution without the use of organic solvents. In a typical embodiment of the process, three separate product fractions, i.e. an arabinose fraction, a galactose fraction and a rhamnose fraction can be recovered in one chromatographic fractionation step. The process may be carried out with fewer process steps than in the known processes for recovering arabinose. The process of the invention also provides pure crystalline arabinose. The crystallization of arabinose may be carried out directly from the hydrolyzed product or from the arabinose-containing fraction obtained from the chromatography or membrane filtration. The crystallization of arabinose in accordance with the present invention preferably comprises a single-stage crystallization resulting in crystalline arabinose with a high purity and with a high yield.

### DEFINITIONS RELATING TO THE INVENTION

In the specification and throughout the claims, the following definitions have been used:

SAC refers to a strongly acid cation exchange resin.

WAC refers to a weakly acid cation exchange resin.

SBA refers to a strongly basic anion exchange resin.

WBA refers to a weakly basic anion exchange resin.

MAX refers to methyl-α-D-xylopyranoside

DVB refers to divinylbenzene.

ACN refers to acetonitrile.

DS refers to a dry substance content measured by Karl Fischer titration, expressed as % by weight.

RDS refers to a refractometric dry substance content, expressed as % by weight.

Purity refers to the content of the compound of interest on DS or RDS.

SS refers to supersaturation in respect of arabinose. It is defined as the ratio of the arabinose concentration in water at the measuring and at solubility points. The solubility refers to a pure arabinose-water solution at the measuring point temperature.

SMB refers to a simulated moving bed process.

DSC refers to differential scanning calorimetry.

### BRIEF DESCRIPTION OF THE DRAWINGS

In the following, the invention will be described in greater detail by means of preferred embodiments and with reference to the attached drawings, in which

FIG. 1 is a graphical presentation of the elution profile obtained from Example 2 (chromatographic fractionation of an arabinose-containing solution derived from gum arabic with a strongly acid cation exchange resin in Ca²⁺ form).

FIG. 2 is a graphical presentation of the elution profile obtained from Example 3 (chromatographic fractionation of an arabinose-containing solution derived from gum arabic with a strongly acid cation exchange resin in Na⁺ form).

FIG. 3 is a graphical presentation of the elution profile obtained from Example 4 [(chromatographic fractionation of an arabinose-containing solution derived from gum arabic with a weakly acid cation exchange resin in H⁺ form (WAC under acidic conditions)].

FIG. 4 is a graphical presentation of the elution profile obtained from Example 5 (chromatographic fractionation of an arabinose-containing solution derived from gum arabic with a weakly acid cation exchange resin in Na⁺ form).

FIG. 5 is a graphical presentation showing the effect of the galactose content of the crystallization feed on the purity of the arabinose crystals (the content of arabinose in the crystals).

FIG. 6 is a graphical presentation showing the effect of the galactose content of the crystallization feed on the melting point of the arabinose crystals.

### DETAILED DESCRIPTION OF THE INVENTION

The invention relates to a process of recovering arabinose and optionally at least one other monosaccharide selected from the group consisting of galactose, rhamnose and mannose from vegetable fiber rich in heteropolymeric arabinose.

The process of the invention comprises the following steps:

(a) controlled acidic or enzymatic hydrolysis of said vegetable fiber in an aqueous solution to hydrolyze more than 50% of the heteropolymeric arabinose present in the vegetable fiber to monomeric arabinose and to produce an aqueous hydrolyzate containing at least 10% arabinose on DS, at least one other monosaccharide selected from the group consisting of galactose and optionally rhamnose and mannose, and optionally poly-, oligo and/or disaccharides, soluble polymers and undissolved solids,

(b) optional neutralization of said aqueous hydrolyzate,

(c) separation of the undissolved solids from said aqueous hydrolyzate obtained in step (a) or from said neutralized hydrolyzate obtained in step (b) to obtain a clarified hydrolysate,

(d) optional fractionation of said clarified hydrolyzate in an aqueous solution to obtain a fraction enriched in arabinose, which comprises at least 50% arabinose and less than 30% of one or more monosaccharides selected from galactose and optionally rhamnose and mannose on DS, at least one other fraction selected from the group consisting of a fraction enriched in galactose and a fraction enriched in rhamnose and a fraction enriched in mannose, and optionally one or more fractions enriched in poly-, oligo- and/or disaccharides and soluble polymers, followed by the recovery of said fraction enriched in arabinose and optionally one or more of said other fractions, and

(e) crystallization of arabinose in an aqueous solution from said hydrolysate obtained in step (c) or from said fraction enriched in arabinose obtained in step (d) to obtain crystalline arabinose having a galactose content of less than 1% on DS.

The fractionation in step (c) typically comprises chromatographic fractionation and/or membrane filtration.

In connection with the present invention, “heteropolymeric arabinose” refers to arabinose which is bound as a constituent to complex heteropolysaccharides. In addition to arabinose, said heteropolysaccharides typically contain galactose, rhamnose, mannose, glucose, galacturonic acid, glucuronic acid, 4-O-methyl glucuronic acid, xylose and ferulic acid, for example. In said heteropolysaccharides, said constituents are bound to each other with different linkages.

In connection with the present invention, “arabinose” refers to monomeric arabinose, which is typically L-arabinose.

In connection with the present invention, “soluble polymers” refer to hemicellulose polymers and pectin, for example.

The starting material in the process of the present invention is vegetable fiber material rich in heteropolymeric arabinose. Said vegetable fiber may be soluble or insoluble in water. In said vegetable fiber, the heteropolymeric arabinose is present in soluble/insoluble polysaccharides, such as araban, galactan and arabinogalactan. In a preferred embodiment of the invention, said vegetable fiber consists of water-soluble or alkali-soluble vegetable fiber.

In connection with the present invention, araban refers to a heteropolysaccharide, which contains arabinose as one constituent of the polysaccharide chain. As other constituents, said araban polysaccharide typically contains at least one other monosaccharide unit typically selected from galactose, xylose and rhamnose. Furthermore, araban may contain other constituents that are recited above for “heteropolymeric arabinose”.

Galactan refers to a heteropolysaccharide which contains galactose as one constituent of the polysaccharide chain. As other constituents, said galactan polysaccharide typically contains galacturonic acid and at least one other monosaccharide, which is selected from arabinose and rhamnose, for example. Furthermore, galactan may contain other constituents that are recited above for “heteropolymeric arabinose”.

Arabinogalactan refers to a heteropolysaccharide consisting of a galactan skeleton containing grafted arabinose groups. Furthermore, arabinogalactan may contain other monosaccharides and other components, such as those recited above for “heteropolymeric arabinose” as minor constituents.

Said araban, galactan and arabinogalactan are preferably water-soluble.

Said vegetable fiber rich in heteropolymeric arabinose typically contains arabinose in an amount of more than 15%, preferably more than 35%, based on the dry substance content (DS) of the vegetable fiber. The galactose content of said vegetable fiber is typically in the range of 5 to 40% on DS.

Exudate gums are especially preferred arabinose sources in the process of the present invention. In connection with the present invention, exudates gums refer to exudates formed in wounds of some tropical trees and bushes. Gum arabic, gum tragacanth and gum ghatti are representative examples of said exudate gums. Gum arabic is recovered from African acacia trees, especially from Acacia senegal, which is cultivated in Sudan. The L-arabinose-content of gum arabic is typically about 35 to 45% on DS and the galactose content about 20 to 40% on DS. Gum tragacanth gum is recovered from the Asian bush species Astragalus and gum ghatti from the Indian Anogeissus tree.

Further examples of useful arabinose sources in the process of the present invention include pectic compounds from sugar beet and chickory root. For example, sugar beet pulp is one useful arabinose source in the present invention. Sugar beet pulp typically contains about 21% arabinose. Further useful arabinose sources include algae, citrus pectin, apple pectin, the araban of citrus fruit and the arabinogalactan of the larch tree as well as hardwood bark, preferably beech or birch bark, grain straw or hulls, corn husks, corn cobs, corn fibers and bagasse.

The first step (a) of the process of the present invention comprises controlled hydrolysis of said vegetable fiber rich in heteropolymeric arabinose in an aqueous solution to hydrolyze more than 50% of the heteropolymeric arabinose present in the vegetable fiber into monomeric arabinose and to provide an aqueous hydrolyzate containing at least 10% arabinose on DS, at least one other monosaccharide selected from the group consisting of galactose and optionally rhamnose and mannose, and poly-, oligo- and/or disaccharides, soluble polymers and undissolved solids. Neutral and acidic poly-, oligo- and disaccharides are typically formed in the hydrolysis in addition to monomeric sugars and organic acids.

The hydrolysis step of the present invention is preferably carried out as a selective hydrolysis by adjusting the hydrolysis conditions (temperature, pH and hydrolysis time) so that an optimal release of arabinose in relation to galactose and other sugars is achieved. The release of arabinose, galactose and rhamnose from the polysaccharide constituents of the vegetable fiber typically takes place in the order: (1) arabinose, (2) galactose, (3) rhamnose.

The hydrolysis is typically carried out as acid hydrolysis with an inorganic acid, such as sulphuric acid, sulphurous acid and hydrochloric acid or with an organic acid, such as acetic acid, formic acid and oxalic acid. Acid concentration in hydrolysis solution is in the range 0.1-5%.

The hydrolysis may also be carried out as enzymatic hydrolysis. The enzymatic hydrolysis is typically effected with galactase and arabinanase enzymes and pectinase enzymes. Enzymes having α-L-arabinofuranosidase activity and endo-hemicellulase activity, such as endo-1,4-β-xylanase activity, can be mentioned as examples of suitable enzymes to be used for the production of monomeric arabinose.

In the selective hydrolysis, the hydrolysis conditions (for example the temperature and the hydrolysis time) are selected so that an optimal release of arabinose from the polysaccharides of the vegetable fiber is achieved. In optimal hydrolysis, the hydrolysis conditions are typically selected so that more than 50%, preferably more than 70%, and most preferably more than 80% of said heteropolymeric arabinose present in the vegetable fiber is hydrolyzed into monomeric arabinose.

In a typical embodiment of the invention, the acid hydrolysis temperature is 70 to 140° C., preferably 90 to 120° C., and the hydrolysis time is in the range of 0.4 to 6 hours. In one embodiment of the invention, the acid hydrolysis is typically carried out at a pH of 0.7 to 2.5, preferably 1 to 1.5. In another embodiment of the invention, said acid hydrolysis is carried out at a temperature in the range of 90 to 100° C. and at a pH in the range of 1.1 to 2.0 and the hydrolysis is continued for 1 to 3 hours. The amount of acid used for the hydrolysis is typically 5 to 15% based on the dry substance content of said vegetable fiber used as the starting material.

The dry substance content of the hydrolyzate is typically 10 to 30% by weight.

In a typical embodiment of the invention, the hydrolysis conditions are selected so as to obtain a hydrolyzate where the content of arabinose is at least 10%, typically more than 20%, more preferably more than 30%, and most preferably more than 45% on DS.

In a preferred embodiment of the invention, the hydrolysis conditions are selected so as to obtain a hydrolyzate where the content of galactose is less than 10%, preferably less than 5% and most preferably less than 2% on DS. In an acid hydrolysis for gum, the hydrolysis conditions in this embodiment of the invention may be selected as follows: the temperature is 90 to 100° C., pH is 1.1 to 2.0, and the hydrolysis time is 1 to 3 h.

The hydrolysis may also be carried out as a total hydrolysis by adjusting the hydrolysis conditions so that essentially all of the hemicellulose components, including arabinose, galactose and rhamnose present in the polysaccharides of the vegetable fiber are released into the hydrolyzate to produce a solution containing essentially all of the arabinose, galactose and rhamnose present in said polysaccharides. In addition to arabinose, the hydrolysis product of the total hydrolysis thus includes essential amounts of galactose and rhamnose and optionally other monosaccharides present as constituents of the araban polysaccharides, such as xylose, mannose and glucose. A typical composition of the total hydrolysis product is for example about 40% arabinose, about 28% galactose and smaller amounts of rhamnose. The hydrolysis product may also include glucuronic acid, 4-O-methyl glucuronic acid and galacturonic acid, as well as acid and neutral di-, poly- and oligosaccharides, for example.

In the total hydrolysis, the acid hydrolysis temperature is typically more than 100° C., preferably 100 to 130° C., and the acid hydrolysis time is in the range of 0.5 to 6 hours. The acid hydrolysis is typically carried out at a pH of 0.5 to 2.5, preferably 1 to 1.5. The amount of the acid used in the hydrolysis may be selected on the basis of the hydrolysis temperature: a lower temperature requires a higher amount of acid and/or a longer reaction time and a higher temperature requires a lower amount of acid and/or a shorter reaction time.

The hydrolysis may be carried out as a batch process or as a continuous process. The hydrolysis vessel may be a mixed reactor or a tubular reactor, optionally provided with a continuous flow. The hydrolysis material may be used in a dry form or in a wet form.

After the hydrolysis, the undissolved solids are separated from the aqueous hydrolyzate in accordance with step (c) in a known manner, such as filtration. A clarified hydrolyzate is obtained.

When the hydrolysis is carried out as an acid hydrolysis, the hydrolysis step is typically followed by neutralization in accordance with step (b). Neutralization may be carried out with any useful alkali, such as CaO, MgO, NaOH, KOH, Na₂CO₃ and CaCO₃.

The reagents used for the hydrolysis and neutralization typically introduce various salts into the hydrolyzate. Said salts are preferably removed from the hydrolyzate in subsequent fractionation steps of the process.

The hydrolysis product containing arabinose and at least one other monosaccharide selected from galactose and optionally rhamnose and mannose and optionally poly-, oligo- and/or disaccharides as well as salts from the acid hydrolysis and neutralization is then subjected to fractionation in accordance with step (d). The fractionation provides a fraction enriched in arabinose, at least one other fraction selected from the group consisting of a fraction enriched in galactose, a fraction enriched in rhamnose and a fraction enriched in mannose and optionally one or more fractions enriched in poly-, oligo- and/or disaccharides and soluble polymers. The fractionation is followed by the recovery of said fraction enriched in arabinose and optionally one or more of said other fractions.

The fraction enriched in arabinose typically contains at least 50% arabinose and less than 30% of one or more monosaccharides selected from galactose and optionally rhamnose and mannose on DS. In an especially preferred embodiment of the process, the fraction enriched in arabinose contains at least 70% arabinose and less than 10% of one or more monosaccharides selected from galactose and optionally rhamnose and mannose on DS.

The chromatographic fractionation of the process of the present invention may be carried out using a column packing material selected from cation and anion exchange resins. The resins are used in a gel form or in a macroporous form. In a preferred embodiment of the invention, said resins are used in a gel form.

In one embodiment of the invention, the chromatographic fractionation is carried out with cation exchange resins. The cation exchange resins may be selected from strongly acid cation exchange resins or weakly acid cation exchange resins.

Said strongly acid cation exchange resins may be in a monovalent cation form or in a divalent cation form. In a preferred embodiment of the invention, said strongly acid cation exchange resin is in H⁺, Na⁺, K⁺, Ca²⁺, Mg²⁺, Sr²⁺, NH₄⁺ and Ba²⁺ form. A resin in Al³⁺ form may also be used (WO 01/21271 A1, Sohkar Oy).

Said strongly acid cation exchange resin may have a styrene skeleton. In a preferred embodiment of the invention, the resin is a sulphonated polystyrene-co-divinylbenzene resin. Other alkenylaromatic polymer resins, like those based on monomers like alkyl-substituted styrene or mixtures thereof, may also be applied. The resin may also be crosslinked with other suitable aromatic crosslinking monomers, such as divinyltoluene, divinylxylene, divinylnaphtalene, divinylbenzene, or with aliphatic crosslinking monomers, such as isoprene, ethylene glycol diacrylate, ethylene glycol dimethacrylate, N,N′-methylene bis-acrylamide or mixtures thereof. The cross-linking degree of the resin is typically from about 1 to about 20%, preferably from about 3 to about 8%, of the cross-linking agent, such as divinyl benzene.

Said weakly acid cation exchange resins may be in a monovalent cation form or in a divalent cation form, preferably in H⁺ or Na⁺ form.

Said weakly acid cation exchange resin is preferably an acrylic cation exchange resin having carboxylic functional groups. However, the resin may be a resin other than an acrylic resin, for example a styrene resin, and the functional groups may be groups other than a carboxylic group, e.g. another weak acid. Such an acrylic resin is preferably derived from methyl acrylate, ethyl acrylate, buthyl acrylate, methylmethacrylate or acrylonitrile or acrylic acids or mixtures thereof. The resin may be crosslinked with a cross-linking agent, e.g. divinylbenzene, or with the other crosslinking agents mentioned above. A suitable cross-linking degree is 1 to 20% by weight, preferably 3 to 8% by weight.

Zeolites can also be used as cation exchange resins in the chromatographic fractionation step of the process of the present invention. Furthermore, alumina is useful in the chromatographic fractionation of the present invention.

In another embodiment of the invention, said chromatographic fractionation is carried out using a column packing material selected from anion exchange resins. Said anion exchange resins may be selected from strongly basic anion exchange resins and weakly basic anion exchange resins.

Said strongly basic anion exchange resins are typically used in HSO₃⁻ or SO₄²⁻ form. Said strongly basic anion exchange resin may have a styrene or an acrylic skeleton. The resin may be crosslinked with divinylbenzene. Other alkenylaromatic polymer resins, like those based on monomers like alkyl-substituted styrene or mixtures thereof, may also be applied. The resin may also be crosslinked with other suitable aromatic crosslinking monomers, such as divinyltoluene, divinylxylene, divinyinaphtalene, divinylbenzene, or with aliphatic crosslinking monomers, such as isoprene, ethylene glycol diacrylate, ethylene glycol dimethacrylate, N,N′-methylene bis-acrylamide or mixtures thereof. The cross-linking degree of the resins is typically from about 1 to about 20%, preferably from about 3 to about 8% of the cross-linking agent, such as divinyl benzene.

Said weakly basic anion exchange resins are preferably weakly basic anion exchange resins having an acrylic skeleton. The weakly basic anion exchange resin is preferably derived from acrylic esters (H₂═CR—COOR′, where R is H or CH₃ and R′ is alkyl group like methyl, ethyl, isopropyl, butyl etc.) like methyl acrylate, ethyl acrylate, butyl acrylate, methyl methacrylate, acrylonitrile or acrylic acids or mixture thereof. The acrylic matrix is crosslinked with a suitable crosslinker which can be, for example, of aromatic type like divinylbenzene (DVB) or of aliphatic type like isoprene, 1,7-octadiene, trivinylcyclohexane, diethylene glycol divinyl ether, N,N′-methylenebisacrylamide, N,N′-alkylene bisacrylamides, ethyleneglycol dimethacrylate and other di-, tri-, tetra-, pentacrylates and pentamethacrylates. A suitable crosslinking degree with divinylbenzene is from 1 to 10 weight-% DVB, preferably from 3 to 8 weight-%. The weakly basic anion resin is manufactured of the crosslinked polyacrylic polymer by amination with suitable amine like mono-, di-, tri-, tetra-, penta- or hexamines or other polyamines. For example dimethylamine, diethylene triamine, triethylene tetramine, tetraethylene pentamine, pentaethylene hexamine and dimethylaminopropylamine are suitable amines.

Another weakly basic anion exchange resin structure is epichlorohydrin-based polycondensation anion exchangers. The chloromethyl and epoxy group of epichlorohydrin react with polyamines, forming crosslinked gel type anion exchangers. For example condensation reaction of epichlorohydrin with triethyleneteramine results following anion resin structure. This type of anion resin contains both weakly basic (tertiary amine) and strongly basic (quaternary ammonium) functional groups.

Another class of weakly basic anion exchange resins is the aminated polycondensation products of phenol and formaldehyde.

Another well known way to produce weakly basic anion exchange resins is aliphatic amines and ammonia polycondensation resins. Cross-linked resin structures are formed when monomeric amines or ammonia are reacted for example with formaldehyde. The reaction between amine and formaldehyde forms methylol and/or azomethine groups, which may further react to form polycondensates. A well-known structure of this type is a reaction resin of formaldehyde, acetone and tetraethylenepentamine. Aromatic amines can also be cross-linked with formaldehyde resulting in a weakly basic anion exchanger.

Different types of cross-linked polyvinylpyridine based ion exchangers having pyridine as the functional group are also useful as weakly base anion exchangers.

Said weakly basic anion exchange resins may be used in OH⁻ form, for example.

The average particle size of the resins which are useful in the present invention is normally 10 to 2000 micrometers, preferably 100 to 400 micrometers. In a preferred embodiment of the invention, the resins are gel-type resins.

Manufacturers of the resins include, for example, Finex Oy, Purolite, Dow Chemicals, Bayer AG and Rohm & Haas Co.

In the chromatographic fractionation operation, the cations/anions of the resin are preferably in substantial equilibrium with the cations/anions of the mobile phase of the system and/or with the feed material of the system.

The eluent used in the chromatographic fractionation is preferably water, but even solutions of salts and water are useful. Furthermore, condensates obtained from the evaporation (concentration) of the product fractions from the chromatographic separation are useful eluents.

The temperature of the chromatographic fractionation is typically in the range of 20 to 90° C., preferably 40 to 65° C. The pH of the solution to be fractionated is typically in the range of 2 to 9.

The chromatographic fractionation may be carried out using all known modifications of the chromatographic fractionation, typically as a batch process or a simulated moving bed process (SMB process). The SMB process is preferably carried out as a sequential or a continuous process.

In the simulated moving bed process, the chromatographic fractionation is typically carried out using 2 to 14 columns connected in series and forming at least one loop. The columns are connected with pipelines. The flow rate in the columns is typically 0.5 to 10 m³/(hm²) of the cross-sectional area of the column. Columns are filled with a column packing material selected from the resins described above. The columns are provided with feed lines and product lines so that the feed solution and the eluent can be fed into the columns and the product fractions collected from the columns. The product lines are provided with on-line instruments so that the quality/quantity of the production flows can be monitored during operation.

During the chromatographic SMB separation, the feed solution is circulated through the columns in the loops by means of pumps. Eluent is added, and the product fraction containing the desired monosaccharide, other optional product fractions and residual fractions are collected from the columns.

In the batch process, the feed solution and the eluent are fed to the top of the column system and the product fractions are collected from the bottom of the system.

Before the chromatographic fractionation, the feed solution may be subjected to one or more pretreatment steps selected from softening by ion-exchange treatment, dilution, concentration e.g. by evaporation, pH adjustment and filtration, for example. Before feeding into the columns, the feed solution and the eluent are heated to the fractionation temperature described above (for instance in the range of 50 to 85° C.).

The chromatographic fractionation provides a fraction enriched in arabinose, at least one other fraction selected from the group consisting of a fraction enriched in galactose, a fraction enriched in rhamnose and a fraction enriched in mannose, and optionally one or more fractions enriched in di-, poly- and/or oligosaccharides.

The arabinose fraction obtained from the chromatographic fractionation typically contains at least 50%, preferably at least 70% and most preferably at least 80% arabinose on DS. Furthermore, the arabinose fraction typically contains less than 30%, preferably less than 10%, more preferably less than 5% and most preferably less than 2% of one or more monosaccharides selected from galactose and optionally rhamnose and mannose on DS as impurities.

The arabinose yield in the arabinose fraction obtained from the chromatographic fractionation is typically at least 50%, preferably more than 70% and more preferably more than 90% on the arabinose present in the hydrolyzate used for the chromatographic fractionation. The removal of galactose, rhamnose and mannose is typically at least 50%, preferably at least 70%, most preferably at least 90% based on the content of these monosaccharides in the hydrolyzate before the fractionation. The chromatographic fractionation of the invention may also comprise recovering glucuronic acid and galactose oligomers and polymers as further product fractions.

To improve the yield of the chromatographic fractionation, recycle fractions of the chromatographic fractionation may also be used.

The chromatographic fractionation method of the invention may further comprise one or more purification steps selected from ion exchange, evaporation and filtration. These purification steps may be carried out before or after said chromatographic fractionation steps.

When the hydrolysis step is carried out as selective hydrolysis to provide a hydrolyzate containing arabinose as a predominant component, the chromatographic fractionation is preferably carried out with a strongly acid cation exchange resin.

When the hydrolysis is carried out as total hydrolysis and the hydrolysis product also contains essential amounts of galactose and rhamnose, rhamnose is preferably separated with a weakly acid cation exchange resin in Na⁺ form and galactose with a weakly acid cation exchange resin in H⁺ form.

Hexose sugars, such as galactose, mannose and glucose may be removed by fermentation, for example with a yeast.

Said fractionation may also be carried out by membrane filtration, which is typically selected from ultrafiltration and nanofiltration. In a preferred embodiment of the invention, the membrane filtration typically comprises nanofiltration. The nanofiltration provides two fractions: a retentate enriched in di-, poly- and/or oligosaccharides and a permeate enriched in arabinose.

The nanofiltration is typically carried out at a pH of 1 to 7, preferably 3 to 6.5, most preferably 5 to 6.5. The pH depends on the composition of the solution to be fractionated and the membrane used for the nanofiltration.

The nanofiltration is typically carried out at a pressure of 10 to 50 bar, preferably 15 to 35 bar. A typical nanofiltration temperature is 5 to 95° C., preferably 30 to 60° C. The nanofiltration is typically carried out with a flux of 10 to 100 l/m² h or with a flux of 2 to 50 l/m² h, depending on the concentration and the viscosity of the nanofiltration feed.

The nanofiltration membrane used in the present invention may be selected from polymeric and inorganic membranes having a cut-off size of 100 to 2500 g/mol, preferably 150 to 1000 g/mol, most preferably 150 to 500 g/mol.

Typical polymeric nanofiltration membranes useful in the present invention include, for example, polyether sulfone membranes, sulfonated polyether sulfone membranes, polyester membranes, polysulfone membranes, aromatic polyamide membranes, polyvinyl alcohol membranes and polypiperazine membranes and combinations thereof.

Typical inorganic membranes include ZrO₂— and Al₂O₃-membranes, for example.

Preferred nanofiltration membranes are selected from sulfonated polysulfone membranes and polypiperazine membranes. For example, specific useful membranes include: Desal-5 DL and Desal-5 DK nanofiltration membrane (manufacturer Osmonics) and NF-270 nanofiltration membrane (manufacturer Dow Deutschland), for example.

The yield of arabinose in the nanofiltration is typically more than 50%, preferably more than 70% and most preferably more than 90% on the arabinose present in the hydrolyzate.

Said fractionation by membrane filtration may further contain one or more purification steps selected from ion exchange, evaporation and filtration. These further purification steps may be carried out before or after said membrane filtration.

In one embodiment of the invention, said fraction enriched in di-, poly- and/or oligosaccharides (which has been obtained from the chromatographic fractionation or from the membrane filtration) may be further subjected to hydrolysis to obtain a hydrolyzate containing galactose and optionally rhamnose and mannose and additional arabinose. Galactose and optionally rhamnose and mannose and additional arabinose may then be separated from the hydrolyzate. The separation is preferably carried out by chromatographic fractionation. Galactose, rhamnose and/or mannose may then be subjected to crystallization.

In one embodiment of the invention, the process comprises at least two fractionations selected from chromatographic fractionation and/or membrane filtration, in any desired sequence.

In one embodiment of the process of the present invention, the process further comprises the recovery of a fraction enriched in soluble polymers in step (d). In a preferred embodiment of this aspect of the invention, the soluble polymers comprise pectin. The separation of pectin may be carried out by nanofiltration, whereby the pectin fraction is recovered as the nanofiltration retentate and the arabinose fraction is recovered as the nanofiltration permeate.

In a further embodiment of the process of the present invention, the process further comprises the separation of xylose from the hydrolyzate as a prefractionation step before the fractionation step (d).

The product fractions obtained from the chromatographic fractionation or membrane filtration are then subjected to crystallization to obtain crystalline arabinose and optionally galactose, rhamnose and/or mannose. It is also possible to subject the hydrolysis product directly to crystallization.

The crystallization of each component may be carried out by traditional methods, such as cooling crystallization in a temperature range of 0 to 80° C. or precipitation crystallization. The crystallization of arabinose may also advantageously be carried out by a boiling crystallization method or by a boiling and cooling crystallization method. To obtain an arabinose product with high purity, the crystallization of arabinose is carried out from a solution where the content of galactose is below critical limits of less than 10%, preferably less than 5%, and most preferably less than 2% on DS.

Any combinations of two or more of said crystallizations may also be used.

The crystallization is typically carried out using a solvent selected from water, alcohol, such as ethanol, or a mixture thereof. In a preferred embodiment of the invention, the crystallization is carried out from water.

The crystallization preferably comprises crystallization of arabinose.

In one embodiment of the invention, the crystallization of arabinose is carried out by cooling crystallization. The solution containing arabinose is first evaporated to an appropriate dry substance content (e.g. to an RDS of about 60 to 80%) depending on the arabinose content of the solution. The slightly supersaturated solution may be seeded with seed crystals of arabinose. The seeds, if used, are pulverized crystals in a dry form or they are suspended in a crystallization solvent, which may be water, an alcohol, such as ethanol, or a mixture thereof. A typical crystallization solvent is water. After seeding, the crystallization mass is subjected to cooling with simultaneous mixing until the crystallization yield or viscosity is optimal for the separation of crystals. Some additional crystallization solvent may be added during cooling to improve the crystallization yield or the crystal separation performance. The crystallization mass may then be mixed at the final temperature for a period of time, preferably 0.5 to 24 hours, to reach the maximum crystallization yield. The crystals are separated from the mother liquor for example by filtration or centrifugation. The filtration cake is washed with the crystallization solvent and optionally dried to obtain a product with a high purity.

In another embodiment of the invention, the crystallization of arabinose is carried out by boiling crystallization combined with cooling crystallization. The solution containing arabinose is first evaporated to slight supersaturation at the boiling point of the solution. The solution is seeded and the evaporation is continued at the boiling point of the crystallization mass (i.e. the mixture of the supersaturated solution and crystals) to obtain improved crystal size distribution and yield, until a crystallization mass is obtained, in which the crystal yield is 1 to 60% on arabinose, and the dry solids content of the mass is over 60% by weight. The evaporation is preferably carried out at a temperature of 50 to 70° C. After boiling crystallization, the crystallization mass is subjected to cooling with simultaneous mixing until the crystallization yield or viscosity is optimal for the separation of crystals. The cooling time is preferably 10 to 60 hours. The temperature drop during cooling is preferably 5 to 40° C., depending on the boiling crystallization yield and the crystal size distribution. Additional crystallization solvent may be added during cooling to further improve the crystallization yield and the crystal separation performance. The crystallization mass may then be mixed at the final temperature for a period of time, preferably 0.5 to 24 hours, to reach maximum crystallization yield. The crystals are separated from the mother liquor for example by filtration or centrifugation. The filtration cake is washed with the crystallization solvent and optionally dried to obtain crystals with high purity.

In the boiling crystallization, the temperature and the supersaturation gradient between the heat carrier surface and the crystallization mass is advantageous. Any small crystals may grow, and the formation of any new crystal nuclei may be avoided. The rate of crystallization is high, since the temperature is suitable and the viscosity of the mother liquor is low, i.e. mass and heat transport are efficient because of boiling. The boiling crystallization makes it easy to control the crystal size. Also, a good output (kg crystals/m³ crystallization mass), a high productivity (kg crystals/h/m³ crystallization mass), a good yield and good crystal quality are achieved. Centrifugation of the mass is easy.

In the precipitation crystallization, the crystallization is essentially carried out by means of nucleation. The precipitation crystallization is preferably carried out at high viscosity and at high supersaturation and it may include boiling and cooling stages, or both.

The precipitation crystallization may be carried out as described in U.S. Pat. No. 5,980,640. In one embodiment of the invention, the precipitation crystallization can be carried out starting from an arabinose purity of more than 35%, preferably more than 45%. The arabinose solution is evaporated to a concentration higher than 75% to bring the solution to sufficient supersaturation to effect nucleation at a temperature of 60 to 70° C. The crystallization mass is then cooled under agitation until the viscosity of the crystallization mass is high, typically over 50 Pas. The agitation is continued at a temperature of 20 to 40° C. until the crystallization has proceeded sufficiently. Thereafter, the viscosity of the crystallization mass is adjusted to an adequate value (10 to 50 Pas) for the separation of the crystals by adding water or optionally an organic solvent. The crystals are then separated by centrifugation or filtration, for example using a pressure filter. The arabinose content of the crystals thus obtained is typically more than 60%, preferably more than 70%. Washing the crystals will produce crystals, which have an increased purity (higher than 75%).

In one embodiment of the invention, the crystallization of arabinose is carried out from a solution having an arabinose purity of more than 20%, more preferably more than 30%, most preferably more than 35% and especially more than 45% on DS. This embodiment may especially be applied to the separation of arabinose directly from the hydrolyzate. The crystallization typically provides a crystalline arabinose product having a purity of more than 60%, preferably more than 70%, most preferably more than 90%, and especially more than 98% on DS.

In a preferred embodiment of the invention, the crystallization of arabinose is carried out from a solution having an arabinose purity of more than 60% on DS. Boiling crystallization is preferably used in this embodiment of the invention.

In a still more preferred embodiment of the invention, the crystallization of arabinose is carried out from a solution having an arabinose purity of more than 70% on DS. This embodiment may be carried out by cooling crystallization, by boiling crystallization or by combined boiling and cooling crystallization. Productivity of 5 to 20 kg/m³/h can be obtained.

In another preferred embodiment of the invention, said crystallization of arabinose is typically carried out in the presence of less than 10%, preferably less than 5% and most preferably less than 2% galactose on DS as impurity. The arabinose purity of the solution is preferably more than 60%, and more preferably more than 70% on DS. The crystallization of arabinose from said arabinose purity in the presence of said impurity profile typically provides crystalline arabinose having a purity of more than 98%, and more preferably more than 99% on DS.

In accordance with a preferred embodiment of the present invention, arabinose crystals having a high purity, arabinose content over 98% on DS, preferably over 99% on DS, and more preferably over 99.5% on DS, and a low galactose content are obtained by one crystallization step (=single-stage crystallization) from a solution having arabinose content over 65% on DS without dissolving and recrystallization steps. Single stage crystallization may comprise boiling and cooling steps but no recrystallization step.

In another embodiment of the invention, the crystallization of arabinose comprises washing as a further step. This embodiment of the invention typically provides arabinose with a purity of more than 99%.

In a further embodiment of the present invention, the crystallization of arabinose comprises a single-stage crystallization. In a preferred embodiment of this aspect of the invention, the crystallization of arabinose comprises boiling crystallization, optionally combined with cooling crystallization. The crystallization is typically carried out from an arabinose purity of more than 70% on DS. The crystallization typically provides crystalline arabinose having a purity of more than 99.5% on DS, where the yield of arabinose is more than 60% and productivity more than 15 kg/m³/h The crystallization is typically carried out in the presence of less than 10%, preferably less than 5% and most preferably less than 2% galactose on DS as an impurity. Furthermore, the crystallization preferably comprises washing the crystals obtained from the crystallization.

The present invention also provides a novel process for the crystallization of arabinose from a biomass-derived solution. The process is characterized in that the crystallization comprises a single-stage boiling crystallization in an aqueous solution from a biomass-derived solution having an arabinose purity of more than 70% on DS. The boiling crystallization may be optionally combined with cooling crystallization.

In a preferred embodiment of this embodiment of the invention, the crystallization of arabinose is carried out in the presence of less than 10%, preferably less than 5%, and most preferably less than 2% galactose on DS as an impurity. The crystallization of arabinose in the presence of less than 10% galactose as an impurity using boiling and cooling crystallization provides arabinose having a purity of more than 98%, preferably more than 99% on DS. In a preferred embodiment of this aspect of the invention, the crystallization provides crystalline arabinose having a purity of more than 99.5% on DS.

The biomass-derived solution used as the starting material in this aspect of the invention is typically a hydrolyzate of any hemicellulose-containing plant-based material, such as softwood or hardwood, hardwood bark, such as beech bark or birch bark, grain straw or hulls or fibers, corn husks, corn cobs, bagasse and sugar beet pulp, including the vegetable fiber materials rich in heteropolymeric arabinose which are mentioned above.

The process of the invention may also comprise a further step of converting arabinose to ribose. Said conversion is typically carried out by epimerization. The starting material for the epimerization may be crystalline arabinose or an arabinose-rich fraction obtained from the chromatographic fractionation or membrane filtration.

In a still further embodiment of the invention, the invention also provides a novel crystalline L-arabinose product based on vegetable fiber, which has a melting point higher than 164° C., preferably higher than 165° C. determined by DSC with a heating rate of 10° C./min, a melting point higher than 158° C. determined by the European Pharmacopeia method and a purity of more than 99.5% on DS. The crystalline L-arabinose in accordance with the present invention is further characterized by low impurity levels, typically by a galactose level of less than 0.5%, preferably less than 0.2% on DS. In a further aspect of this aspect of the invention, the novel crystalline L-arabinose is characterized by being obtainable by boiling crystallization of arabinose, optionally combined with cooling crystallization.

The novel crystalline L-arabinose of the invention is typically obtained by boiling crystallization from a hydrolyzate derived from vegetable fiber. The hydrolyzate used for the crystallization has a typical arabinose purity of more than 70% on DS and a typical galactose content of less than 5%, preferably less than 2% on DS. The boiling crystallization may be combined with cooling crystallization. The crystallization is preferably carried out by single-stage crystallization.

The invention also relates to the use of the crystalline L-arabinose of the invention in pharmaceutical products and foodstuffs, especially in diet foods and diabetic foods.

The following examples represent illustrative embodiments of the invention without limiting the invention in any way. In the examples, unless otherwise mentioned, arabinose and rhamnose refer to L-arabinose and L-rhamnose, respectively, and references to other sugars (such as galactose) refer to said sugar in D-form.

### EXAMPLE 1

Total Hydrolysis of Various Gum Arabic and Gum Ghatti Samples

The following gum arabic samples were subjected to hydrolysis with H₂SO₄:

1. Gum Arabic, spray dried (Merck 4228.1000)

2. Gum Seyal (Valspray F ref. 25500 (Valmar S. A.))

3. Valcoat VM/960 (Valmar S. A.)

4. Arabic gum Kibbled 56080 (Valmar S. A.)

5. Gum Acacia Seyal, Kibbled (Valmar S. A.)

Gum arabic sample No. 4 was milled with a hammer mill and screened with a sieve (1 mm). The other gum arabic samples were used as they were. The samples were hydrolyzed at a dry solids concentration of about 5% at a pH of 1 at various temperatures for one to six hours, cooled to room temperature and subjected to analysis.

The hydrolysis conditions (hydrolysis time and temperature) and the carbohydrate composition of the hydrolysis products after the hydrolysis (expressed as % on the oven-dried (105° C.) dry substance of the gum arabic) are presented in the following table (“oligosaccharides” refer to di- and oligosaccharides):

| | Sample no | | | | |
|---|---|---|---|---|---|
| | 1 | 2 | 3 | 4 | 5 |
| Hydrolysis | Hydrolysis | | | | |
| products | 120 C./1 h | 100 C./6 h | 120 C./1 h | 100 C./6 h | 120 C./1 h |
| arabinose* | 30 | 45 | 45 | 29 | 43 |
| galactose* | 30 | 34 | 32 | 42 | 29 |
| rhamnose* | 15 | 5 | 4 | 12 | 3.5 |
| xylose* | 2 | 0.5 | 0 | 1 | |
| glucuronic | 5 | 1 | 2 | 4 | 1 |
| acid*** | | | | | |
| oligosac- | 1 | 2.5 | 2 | 1 | 3.9 |
| charides** | | | | | |

*HPLC, amino column
**HPLC-Na⁺(SAC)
***Dionex, PED

The amount of arabinose in the hydrolysis product corresponds to more than 90% of the amount of arabinose in the starting gum arabic sample.

Furthermore, the following table shows the results of a more detailed analysis of gum arabic sample No. 5 above (“oligosaccharides” refer to di- and oligosaccharides):

| | |
|---|---|
| DS content (oven-dried at 105° C.) | 89.1 w-% |

Carbohydrates after hydrolysis (% on oven-dried DS of gum arabic)

| | |
|---|---|
| oligosaccharides** | 3.9 |
| rhamnose* | 3.5 |
| galactose* | 29 |
| arabinose* | 43 |
| mannose* | 0.1 |
| glucose* | 0.7 |
| xylose* | — |
| MAX* | 0.8 |

Cations

| | |
|---|---|
| Ash | 3.5 |
| Ca | 1.2 |
| K | 0.3 |
| Mg | 0.2 |
| Na | 80*** |
| Fe | 20*** |
| N | 0.1 |

*HPLC, amino column
**HPLC-Na⁺(SAC)
(***expressed in ppm)

Furthermore, a gum ghatti sample (supplied from Megamic Globus Est., India) was subjected to hydrolysis in the same way as the gum arabic samples above. The gum ghatti sample was milled with a hammer mill and screened with a sieve (1 mm). The milled gum ghatti sample was hydrolyzed at a dry solids concentration of about 5% at a pH of 1 at various temperatures for 30 to 90 minutes, cooled to room temperature and subjected to analysis.

The following table shows the carbohydrate composition of the hydrolyzed gum ghatti sample:

Carbohydrates after Hydrolysis (HPLC with Amino Column), Oligosaccharides (Na⁺ SAC)

| Hydrolysis | % on DS of gum ghatti | | | | | | |
|---|---|---|---|---|---|---|---|
| conditions | Arabinose | Galactose | Rhamnose | Mannose | Xylose | Glucose | Oligosacch. |
| 110 C./30 min | 48.0 | 9.8 | 1.2 | 0.7 | 2.5 | 1.1 | 8 |
| 130 C./30 min. | 44.0 | 22.0 | 1.1 | 7.2 | 2.3 | 0.9 | 1 |
| 110 C./90 min. | 49.0 | 16.9 | 1.2 | 1.5 | 2.7 | 1.1 | 2 |
| 130 C./90 min. | 36.3 | 21.7 | 1.0 | 11.4 | 1.7 | 1.0 | 1 |
| 120 C./60 min | 45.7 | 19.4 | 1.1 | 3.7 | 2.4 | 1.1 | 1 |

The amount of arabinose in the hydrolysis product corresponds to more than 85% of the amount of arabinose in the starting gum arabic sample.

### EXAMPLE 2

Selective Hydrolysis of Gum Arabic and Chromatographic Fractionation of the Gum Arabic Hydrolyzate with a Strongly Acid Cation Exchange Resin in Ca²⁺ Form.

(A) Preparation of the Gum Arabic Hydrolyzate:

9.25 kg of Gum Arabic Seyal (Valspray, Valmar S/A) was poured into 40 liters of water in a batch reactor. The solution was solubilized over night under agitation. The pH of the gum solution thus obtained was adjusted to 1.07 with 4 kg of 20% H₂SO₄ and the solution was heated to 95° C. The temperature of the solution was maintained at 94 to 96° C. with gentle agitation. Then the reaction was stopped by cooling the solution to 60° C., followed by neutralizing the solution with 3.12 kg of 20% Ca(OH)₂ slurry to a pH of 3.4. The solution was filtered with a Buchner funnel and paper using diatomaceous earth as a filtering aid.

The sugar content of the hydrolyzate was determined at various stages of hydrolysis as well as after the neutralization. The levels of rhamnose, arabinose and galactose (expressed in % on DS of the gum arabic) in the hydrolyzate are presented in the following table, the rest mainly being salts and di-, oligo- and polysaccharides. The amount of arabinose in the hydrolysis product corresponds to more than 85% of the amount of arabinose in the gum arabic sample.

| | Arabinose, | Galactose, | Rhamnose, |
|---|---|---|---|
| Time, h | % on DS | % on DS | % on DS |
| 0.0 | 0.0 | 0.0 | 0.2 |
| 1.1 | 34.8 | 7.9 | 2.5 |
| 2.0 | 39.9 | 5.0 | 3.1 |
| 3.0 | 41.6 | 4.7 | 3.3 |
| 4.0 | 42.3 | 6.9 | 3.4 |
| 5.0 | 42.2 | 8.5 | 3.4 |
| 5.9 | 42.0 | 9.4 | 3.4 |
| After neutralization | 42.3 | 10.4 | 3.5 |

(B) Chromatographic Fractionation of the Gum Arabic Hydrolyzate:

The feed solution for the chromatographic fractionation was the neutralized gum arabic hydrolyzate obtained in accordance with the hydrolysis described above. Before the chromatographic fractionation, the hydrolyzate was subjected to evaporation and filtration. The feed solution had a pH of 3.4 and the following composition (% on RDS):

| | |
|---|---|
| Arabinose | 34.1 |
| Galactose | 9.0 |
| Rhamnose | 2.7 |
| Others | 54.2 |

The solution having the composition presented above was subjected to chromatographic separation. The separation was performed in a pilot scale chromatographic separation column as a batch process. The column with a diameter of 0.225 m was filled with a strongly acid cation exchange resin (Finex CS 11 GC, 5.5% DVB). The height of the resin bed was approximately 5.0 m. The average particle size of the resin was 0.33 mm. The resin was regenerated to a calcium (Ca²⁺) form. The temperature of the column and feed solution and eluent water was 60° C. The flow rate in the column was adjusted to 30 l/h.

The chromatographic separation was carried out as follows:
Step 1: The dry substance of the feed solution was adjusted to 35 g dry substance in 100 g solution according to the refractive index of the solution.
Step 2: 15 l of preheated feed solution was pumped to the top of the resin bed.
Step 3: The feed solution was eluted downwards in the column by feeding preheated ion-exchange water to the top of the column.
Step 4: 50 ml samples of the out-coming solution were collected at 5 min intervals. The composition of the samples was analyzed with HPLC equipment with a refractive index detector and an amino column using a mixture of water with 79% acetonitrile as the eluent.

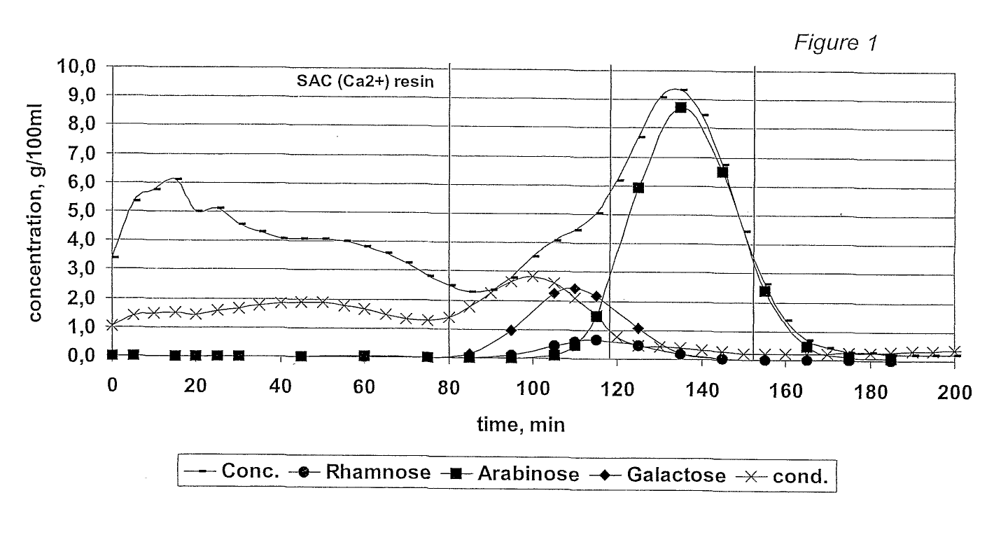

The separation profile is presented in FIG. 1. Elution begins with poly-, oligo- and disaccharides. After these, the elution order of the monosaccharides is galactose, rhamnose and arabinose. Since arabinose elutes later than the others, arabinose in the gum arabic matrix can be effectively separated from galactose with a strongly acid cation exchange resin in a calcium form. For example, galactose and arabinose fractions presented in the table below may be collected in addition to residual fractions. The yield of a component in a fraction is presented in relation to the total amount of that component in all out-coming fractions, calculated from the analysis of the elution profile samples.

| | Galactose fraction | Arabinose fraction |
|---|---|---|
| Volume, I | 19 | 23 |
| Concentration, g/100 ml | 3.8 | 5.6 |
| Composition, % on RDS | | |
| Arabinose | 16 | 88 |
| Galactose | 39 | 5 |
| Rhamnose | 9 | 3 |
| Others | 35 | 4 |
| Yield, % | | |
| Arabinose | 9 | 90 |
| Galactose | 81 | 18 |

The pH of the effluent (e.g. the out-coming solution) was 3.0 to 4.3.

### EXAMPLE 3

Chromatographic Fractionation of a Gum Arabic Hydrolyzate with a Strongly Acid Cation Exchange Resin in Na⁺ Form

The feed solution for the separation was a gum arabic hydrolyzate prepared in accordance with Example 2(A). The hydrolyzate, which mainly contained arabinose, galactose and rhamnose, had been neutralized with Ca(OH)₂ and NaOH and filtered with diatomaceous earth.

The feed solution had the following composition (% on RDS):

| | |
|---|---|
| Arabinose | 30.9 |
| Galactose | 2.3 |
| Rhamnose | 1.7 |
| Others | 65.1 |

The solution having the composition presented above was subjected to chromatographic separation. The separation was performed in a pilot scale chromatographic separation column as a batch process. The column with a diameter of 0.2 m was filled with a strongly acid cation exchange resin (5.5% DVB). The height of the resin bed was approximately 7.95 m. The average particle size of the resin was 0.35 mm. The resin was regenerated into a sodium (Na⁺) form. The temperature of the column and feed solution and eluent water was 60° C. The flow rate in the column was adjusted to 60 l/h. The pH of the feed solution was 5.9.

The chromatographic separation was carried out as follows:
Step 1: The dry substance of the feed solution was adjusted to 30 g dry substance in 100 g solution according to the refractive index (RI) of the solution.
Step 2: 25 l of preheated feed solution was pumped to the top of the resin bed.
Step 3: The feed solution was eluted downwards in the column by feeding preheated ion-exchanged water to the top of the column.
Step 4: 50 ml samples of the out-coming solution were collected at 5 min intervals. The composition of the samples was analyzed with HPLC (Na⁺ SAC) equipment, water was used as the eluent.

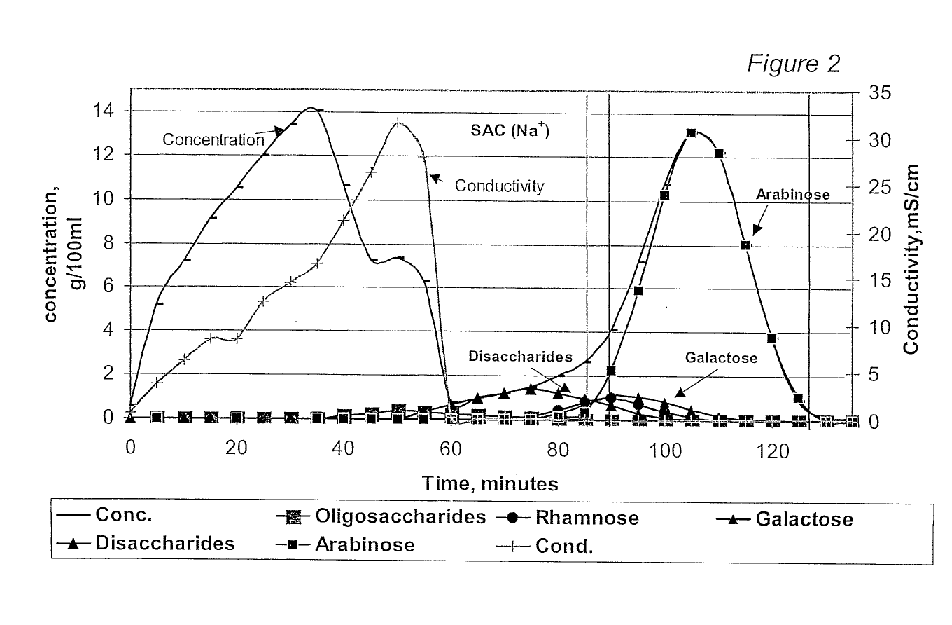

The separation profile is presented in FIG. 2. The series is collected from the third feed. Elution begins with poly-, oligo- and disaccharides. Also salts (corresponding to the conductivity peak) are eluted in the beginning. After these, the elution order of monosaccharides is galactose, rhamnose and arabinose. Since arabinose elutes later than the others, with a sodium-form strongly acid cation exchange resin arabinose can be effectively separated from gum arabic matrix. For example, galactose and arabinose fractions presented in the table below may be collected in addition to residual fractions. The yield of a component in a fraction is presented in relation to the total amount of that component in all out coming fractions, calculated from the analysis of the elution profile samples.

| | Galactose fraction | Arabinose fraction |
|---|---|---|
| Volume, I | 5 | 35 |
| Concentration, g/100 ml | 4 | 7.7 |
| Compositions, % on RDS | | |
| Arabinose | 46 | 93 |
| Galactose | 22 | 4 |
| Rhamnose | 21 | 2 |
| Disaccharides | 11 | 2 |
| Others | 0 | 0 |
| Yield, % | | |
| Arabinose | 4 | 96 |
| Galactose | 22 | 54 |
| Rhamnose | 27 | 31 |
| Disaccharides | 18 | 38 |

The pH of the effluent (e.g. the out-coming solution) was 4 to 7.

### EXAMPLE 4

Chromatographic Fractionation of a Gum Arabic Hydrolyzate with a Weakly Acid Cation Exchange Resin in H⁺ Form and Crystallization of Galactose from the Galactose Fraction

The feed solution for the separation was a gum arabic hydrolyzate prepared in accordance with Example 2(A). The hydrolyzate, which mainly contained arabinose, galactose and rhamnose, had been neutralized with Ca(OH)₂ and NaOH and filtered with diatomaceous earth.

The feed solution had the following composition (% on RDS):

| | |
|---|---|
| Arabinose | 37.8 |
| Galactose | 16.9 |
| Rhamnose | 2.4 |
| Others | 42.9 |

The solution having the composition presented above was subjected to chromatographic separation. The separation was performed in a pilot scale chromatographic separation column as a batch process. The column with a diameter of 0.2 m was filled with a weakly acid cation exchange resin (Finex CA 16 GC, 8% DVB). The height of the resin bed was approximately 15.8 m. The average particle size of the resin was 0.308 mm. The resin was regenerated into hydrogen (H⁺) form with 5% HCl. The temperature of the column, the feed solution and the eluent water was 60° C. The flow rate in the column was adjusted to 60 l/h. The pH of the feed solution was 4.

The chromatographic separation was carried out as follows:
Step 1: The dry substance of the feed solution was adjusted to 45 g dry substance in 100 g solution according to the refractive index (RI) of the solution.
Step 2: 28 l of preheated feed solution was pumped to the top of the resin bed.
Step 3: The feed solution was eluted downwards in the column by feeding preheated ion-exchanged water to the top of the column.
Step 4. 5 ml samples of the out-coming solution were collected at 5 min intervals. The composition of the samples was analyzed with HPLC equipment provided with a refractive index detector and an Na⁺ SAC column (water was used as the eluent).

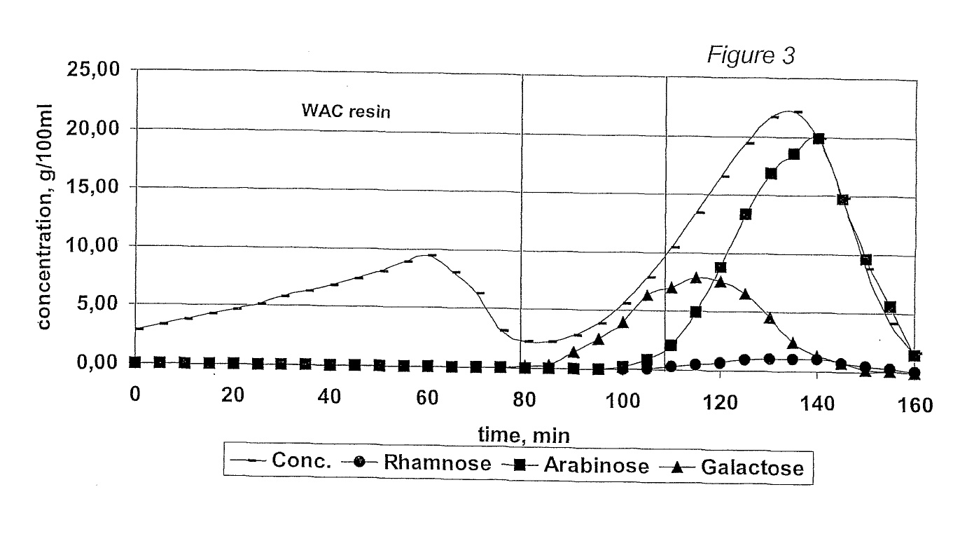

The separation profile is presented in FIG. 3. Elution begins with salts and poly-, oligo- and disaccharides, followed by the elution of monosaccharides in the order: galactose, arabinose and rhamnose. Since galactose elutes earlier than the others, galactose and arabinose can thus be separated from gum arabic matrix with a weakly acid cation exchange resin in acidic conditions. For example, galactose and arabinose fractions presented in the following table may be collected in addition to residual fractions. The yield of a component in a fraction is presented in relation to the total amount of that component in all out-coming fractions, calculated from the analysis of the elution profile samples.

| | Galactose fraction | Arabinose fraction |
|---|---|---|
| Volume, I | 22 | 65 |
| Concentration, g/100 ml | 4.7 | 11.1 |
| Composition, % on RDS | | |
| Arabinose | 5 | 70 |
| Galactose | 66 | 24 |
| Rhamnose | 0 | 4 |
| Others | 29 | 2 |
| Yield, % | | |
| Arabinose | 1 | 99 |
| Galactose | 28 | 71 |

The pH of the effluent (i.e. the out-coming solution) was 2.7 to 4.6.

Galactose is crystallized from the galactose fraction having a galactose content of 66% on DS as follows:

The galactose fraction obtained above is evaporated to RDS of 72% and moved to a 10-liter cooling crystallizer at a temperature of 70° C. Seeding (at 70° C., an RDS of 72%) is made to a boiling syrup with 0.05% galactose seed crystals on DS.

The mass is cooled down from the temperature of 70° C. to a temperature of 20° C. The galactose crystals are separated after 50 hours from seeding by centrifugation. The yield of galactose is about 65%.

The crystals of the first crystal crop thus obtained are dissolved in water to obtain a galactose syrup having a DS of 18%. The syrup is evaporated to an RDS of 63% and moved to a 2-liter reaction vessel at a temperature of 70° C. Seeding (at 70° C., an RDS of 63%) is made to a boiling syrup with 0.02% seeds on DS.

The mass is cooled down from the temperature of 70° C. to a temperature of 20° C. The galactose crystals are separated after 40 hours from seeding by centrifugation. The galactose crystals thus obtained are dried in an oven at a temperature of 60° C. for 12 hours. The galactose yield is about 65%.

### EXAMPLE 5

Chromatographic Fractionation of a Gum Arabic Hydrolyzate with a Weakly Acid Cation Exchange Resin in Na⁺ Form

The feed solution for the separation was a gum arabic hydrolyzate prepared in accordance with Example 2(A). The hydrolyzate, which mainly contained arabinose, galactose and rhamnose, had been neutralized with Ca(OH)₂ and NaOH and filtered with diatomaceous earth.

The feed solution had the following composition (% on RDS):

| | |
|---|---|
| Arabinose | 38.5 |
| Galactose | 17.3 |
| Rhamnose | 2.5 |
| Others | 41.7 |

The solution having the composition presented above was subjected to chromatographic separation. The separation was performed in a pilot scale chromatographic separation column as a batch process. The column with a diameter of 0.225 m was filled with a weakly acid cation exchange resin (Finex CA 16 GC, 8% DVB). The height of the resin bed was approximately 5.2 m. The average particle size of the resin was 0.308 mm. The resin was regenerated into a sodium (Na⁺) form. The temperature of the column, the feed solution and the eluent water was 60° C. The flow rate in the column was adjusted to 30 l/h.

The chromatographic separation was carried out as follows:
Step 1: The dry substance of the feed solution was adjusted to 33 g dry substance in 100 g solution according to the refractive index (RI) of the solution.
Step 2: 14 l of preheated feed solution was pumped to the top of the resin bed.
Step 3: The feed solution was eluted downwards in the column by feeding preheated ion-exchanged water to the top of the column.
Step 4. 50 ml samples of the out-coming solution were collected at 5 min intervals. The composition of the samples was analyzed with HPLC equipment provided with a refractive index detector and a Na⁺ SAC column (water was used as the eluent).

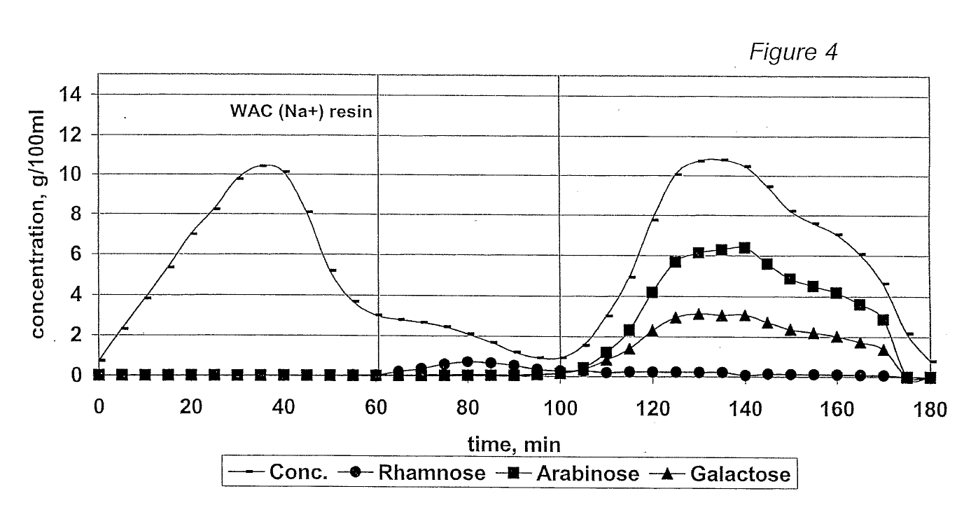

The separation profile is presented in FIG. 4. Elution begins with poly-, oligo- and disaccharides. The elution of monosaccharides starts with rhamnose, which is separated almost completely from the other monosaccharides, followed by arabinose and galactose. Since rhamnose elutes earlier than the others, rhamnose can thus be separated from gum arabic matrix with a sodium-form weakly acid cation exchange resin. For example, rhamnose and arabinose fractions presented in the following table may be collected in addition to residual fractions. The yield of a component in a fraction is presented in relation to the total amount of that component in all out-coming fractions, calculated from the analysis of the elution profile samples.

| | Rhamnose fraction | Arabinose fraction |
|---|---|---|
| Volume, I | 19.5 | 39 |
| Concentration, g/100 ml | 1.7 | 6.5 |
| Composition, % on RDS | | |
| Arabinose | 4 | 57 |
| Galactose | 5 | 29 |
| Rhamnose | 26 | 2 |
| Others | 65 | 12 |
| Yield, % | | |
| Arabinose | 0 | 99 |
| Rhamnose | 61 | 36 |

The pH of the effluent (i.e. the out-coming solution) was 8.0 to 9.7.

### EXAMPLE 6

Chromatographic Fractionation of a Solution Containing Arabinose, Galactose and Rhamnose with a Weakly Basic Anion Exchange Resin in SO₄²⁻ Form

The feed solution for the separation was an arabinose-containing side stream separated from the process disclosed in WO 02/27039 (=U.S. 2002/120135), a process where rhamnose is recovered from a spent sulphite pulping liquor after the recovery of xylose. The feed solution had the following composition (% on RDS):

| | |
|---|---|
| Arabinose | 1.1 |
| Galactose | 4.8 |
| Rhamnose | 20.5 |
| Others | 73.6 |

The solution having the composition presented above was subjected to chromatographic separation. The separation was performed in a pilot scale chromatographic separation column as a batch process. The column with a diameter of 0.1 m was filled with a weakly basic anion exchange resin (Finex AA545GC, 4% DVB). The resin had a methacrylate-DVB skeleton and it had been aminolyzed with dimethylaminopropylamine. The height of the resin bed was approximately 1.4 m. The average particle size of the resin was 0.39 mm. The resin was regenerated into a sulphate (SO₄²⁻) form. The temperature of the column, the feed solution and the eluent water was 50° C. The flow rate in the column was adjusted to 43 ml/min.

The chromatographic separation was carried out as follows:
Step 1: The dry substance of the feed solution was adjusted to 30 g dry substance in 100 g solution according to the refractive index (RI) of the solution.
Step 2: 800 ml of preheated feed solution was pumped to the top of the resin bed.
Step 3: The feed solution was eluted downwards in the column by feeding preheated ion-exchanged water to the top of the column.
Step 4: 50-ml samples of the out-coming solution were collected at 3 min intervals. The composition of the samples was analyzed with Dionex HPLC equipment provided with a pulsed electrochemical detector and a CarboPac PA1® anion exchange column (0.2 M NaOH and water were used as the eluent).

The elution order of the monosaccharides is presented in the following table which shows the retention time and the relative retention of rhamnose, arabinose and galactose. Relative retention is calculated against the retention time of rhamnose. It can be seen from the table that rhamnose elutes earlier than galactose and arabinose.

| | Retention time (min) | Relative retention |
|---|---|---|
| Rhamnose | 54 | 1.00 |
| Arabinose | 66 | 1.22 |
| Galactose | 63 | 1.17 |

The pH of the effluent (the out-coming solution) was 3.5 to 4.4.

### EXAMPLE 7

Crystallization of Arabinose from Water

The starting material for the crystallization was a fraction enriched in arabinose, obtained in accordance with Example 2(B). The starting solution was evaporated at reduced pressure from 5.4% to 74.0% RDS and 1644 grams of the syrup was transferred into a 2000 ml cooling crystallizer and mixed at 71° C. The composition of the crystallization syrup was 88.2% arabinose, 2.9% rhamnose, 6.0% galactose, 0.1% mannose and fucose on RDS, measured by HPLC (the resins in an amino form, +55° C., 79% ACN with 50% H₃PO₄ 6 ml/l). The pH of the syrup was 4.6 and the colour value was 1670 ICUMSA. The seeding was made with 0.15 grams of dry seeds (purity >99%) at 71.0° C. and RDS 68.8%, which corresponds to supersaturation of 1.07. After seeding, the mass was gradually cooled to 25.3° C. in 41 hours. Crystallization yield and supersaturation were measured from the concentrated mother liquor during cooling. The course of the crystallization is presented in the following table:

| Time | Temp. | RDS | SS | | |
|---|---|---|---|---|---|
| hour | ° C. | (m.l) | — | Yield % of arabinose | |
| 0 | 71.0 | 68.8 | 1.07 | 0 | Seeding point |
| 17 | 46.0 | 57.8 | 1.02 | 43 | |
| 22.5 | 40.3 | 56.6 | 1.07 | 46 | |
| 24 | 38.3 | 55.3 | 1.04 | 50 | |
| 41 | 25.3 | 51.5 | 1.08 | 59 | Centrifuging tests |

Centrifuging tests were made with the laboratory basket centrifuge Roto Silenta II (20 min/3500 rpm) with 100 ml and 200 ml washing water. The results are presented in the following table:

| | Test 1 | Test 2 |
|---|---|---|
| Washing water, ml | 100 | 200 |
| Centrifuged mass | | |
| Weight, g | 744 | 739 |
| RDS, % | 68.6 | 68.6 |
| Purity % on DS | 88.2 | 88.2 |
| Obtained crystals | | |
| Weight, g | 275 | 255 |
| RDS, % | 94.1 | 93.7 |
| Purity % on DS | 99.0 | 99.4 |
| Yield, % of arabinose | 56.9 | 53.1 |

Some of the wet crystal samples were dried at 40° C. overnight. The analysis results of the dry crystals are presented in the following table:

| Dry crystal | DS | Colour | Arabinose | Galactose |
|---|---|---|---|---|
| Sample | % | Icumsa | % on DS | % on DS |
| 100 ml wash | 99.7 | 84 | 99.3 | 0.7 |
| 200 ml wash | 99.8 | 30 | 99.3 | 0.7 |

Furthermore, thermal behavior of the washed dry crystals obtained from Test 2 was measured by differential scanning calorimeter (Mettler FP84HT) by using 10° C./min heating rate from 30° C. to 200° C. There was only one peak in the thermogram and the peak temperature was 162.1° C.

A crystal purity of more than 99% was achieved with a good yield with a single stage crystallization. This example demonstrates that high purity arabinose crystals could be obtained by crystallization from a water solution, when the galactose content of the feed liquid was below a critical value of 10% on RDS. It was surprisingly found that galactose is easily included with the arabinose crystals during the crystallization of arabinose even at a low galactose concentration, which, as a rule, has made it difficult to make arabinose crystals with a purity of more than 99%.

### EXAMPLE 8

Epimerization of the Arabinose Fraction Obtained from Chromatographic Fractionation of Example 2(B)

The arabinose fraction obtained from the chromatographic fractionation as described in Example 2(B) was epimerized in a laboratory scale stirred reaction vessel. The volume of the reaction vessel was 1 l and it was provided with a heating jacket. The concentration of the solution was adjusted to 14 g/100 g, and 900 ml of this solution having about 87% on DS of L-arabinose was transferred to the reaction vessel. 1.36 g of MoO₃ was used as the epimerization catalyst and the reaction time at 96° C. was 2 hours. The pH was adjusted to be 2.9 at the end of the reaction. About 29.5% of the available arabinose was converted in the reaction. 78% of the reacted arabinose was converted to L-ribose. The resulting epimerized solution had an L-ribose content of about 20% on DS.

### EXAMPLE 9

Epimerization of Crystalline Arabinose

Crystalline L-arabinose obtained from the crystallization of Example 7 was dissolved in water to obtain 900 ml of a solution having an arabinose concentration of 13.2 g/100 g. The solution was transferred to a laboratory scale stirred reaction vessel. The volume of the reaction vessel was 1 l and it was provided with a heating jacket. 1.26 g of MoO₃ was weighed as a catalyst for the epimerization reaction. At the beginning of the reaction, the pH was adjusted to 3.4 with NaOH. The epimerization was carried out at a temperature of 96° C. for 2 hours. About 33% of the arabinose was converted during the reaction. 76% of the reacted arabinose was converted to L-ribose. The resulting epimerized solution had an L-ribose content of about 25% on DS.

### EXAMPLE 10

Hydrolysis of Gum Arabic Followed by Fractionation with Nanofiltration

(A) Hydrolysis of Gum Arabic

39 l of water was heated in a reactor to 70° C. and 1.05 kg 93% H₂SO₄ was added to the reactor. 15 kg of Gum Acacia Seyal (Kibbled) was added to the reactor resulting in a solution having a DS of 25%. Hydrolysis was started by heating the solution to 95° C. in 15 minutes. The hydrolysis was then continued for 1 h 40 min with gentle agitation while keeping the temperature in the range of 94 to 97° C. The hydrolysis was stopped by cooling the solution to 40° C. and neutralizing with 20% Ca(OH)₂ to a pH of 5. The solution was filtered with a Seitz filter press and paper filter using diatomaceous earth as a filter aid to remove the insoluble substance. The hydrolyzate thus obtained had a DS of 24%. The sugar content of the hydrolyzate (expressed in % on DS) is presented in the following table:

| | |
|---|---|
| Arabinose | 32.0 |
| Galactose | 1.2 |
| Rhamnose | 1.9 |
| Others* | 64.9 |

*mainly salts and oligomers

(B) Nanofiltration of the Hydrolyzate

The solution obtained from the hydrolysis above was subjected to nanofiltration. The nanofiltration was performed in a DSS LabStak 20 membrane unit equipped with 10 pieces of Desal 5 DL membranes (manufactured by Osmonics), (each 0.018 m²). 50 kg of the hydrolyzate after filtering with a Seitz filter was fed to the circulation tank of the nanofiltration equipment and heated to 40° C. The nanofiltration was performed at an inlet pressure of 40 bar at a temperature of 40° C. The nanofiltration was continued by concentration mode, until 40 kg of permeate was collected. The permeate thus obtained had a DS of 10% and about 92% of arabinose was recovered in the permeate. The sugar content of the permeate (expressed in % on DS) is presented in the following table.

| | |
|---|---|
| Arabinose | 88.6 |
| Galactose | 2.8 |
| Rhamnose | 4.4 |
| Others* | 4.2 |

*mainly salts and oligomers

### EXAMPLE 11

Hydrolysis of Sugar Beet Pulp Followed by Chromatographic Fractionation with a Strongly Acid Cation Exchange Resin in Na⁺ Form

(A) Hydrolysis of Sugar Beet Pulp

1100 ml of water was added to a reactor, followed by acidification with 184 g of 18.6% H₂SO₄ to a pH of 0.6. The acidified solution was heated to 95° C. The hydrolysis was started by adding 100 g of dried sugar beet pulp (DS 92%) to the reactor. The hydrolysis was continued for 4 hours while maintaining the temperature in the range of 94 to 96° C. The hydrolysis was stopped by cooling the solution in an ice bath. Biomass was separated with filter paper on a Büchner funnel. In the first step, 880 ml of the filtrate was collected. The arabinose content of the filtrate was 1.4% by weight (analyzed by HPLC). The yield of sugars in the hydrolyzate is presented in the following table (expressed in % on DS of sugar beet pulp.

| | |
|---|---|
| Arabinose | 13.4 |
| Galactose | 2.2 |
| Glucose | 1.6 |

(B) Chromatographic Fractionation of a Sugar Beet Pulp Hydrolyzate with a Strongly Acid Cation Exchange Resin in Na⁺ Form

The feed solution for the chromatographic fractionation was the sugar beet pulp hydrolyzate prepared in accordance with Example 11(A). The composition of the feed solution (having a pH of 3) is presented in the following table (% on DS). ‘Others’ mainly refer to poly-, oligo- and/or disaccharides.

| | |
|---|---|
| Arabinose | 22.0 |
| Galactose | 3.6 |
| Glucose | 2.6 |
| Others | 71.8 |

The chromatographic fractionation was carried out in a chromatographic separation column as a batch process. The column with a diameter of 0.095 m was filled with a strongly acid cation exchange resin (Finex, 4% DVB). The height of the resin bed was approximately 1.68 m. The average particle size of the resin was 0.250 mm. The resin was regenerated into a sodium (Na⁺) form. The temperature of the column, the feed solution and the eluent water were 60° C. The flow rate in the column was adjusted to 50 ml/h. The feed size was 725 ml and the pH of the feed solution was 3.0.

The composition of the arabinose fraction, which was collected from the chromatographic separation, is presented in the following table.

| Composition, % on RDS | |
|---|---|
| Arabinose | 87 |
| Galactose | 6 |
| Glucose | 4 |
| Others | 3 |
| Yield, % | |
| Arabinose | 90 |

(C) Alternative Nanofiltration

Alternatively, the hydrolyzate obtained in example 11(A) is subjected to nanofiltration in accordance with example 10(B) at a pH in the range of 3 to 6. The nanofiltration provides the arabinose fraction as the nanofiltration permeate and another fraction including pectin as the nanofiltration retentate.

### EXAMPLE 12

Hydrolysis of Gum Ghatti

640 ml of water in a 2000 ml glass reactor was heated to 70° C. 300 g of gum ghatti (supplied from Megamic Globus Est., India; DS 87.8%) was slowly added. The gum ghatti was allowed to solubilize for 30 minutes under agitation. The solution was heated to 95° C., followed by the addition of 60 g of H₂SO₄ (18.6%). The hydrolysis was continued for 5 hours with slow agitation at a temperature of 95° C. The hydrolysis was stopped by cooling the solution in an ice bath. The sugar content of the hydrolysate was determined. The results are presented in the following table (expressed in % on DS). ‘Others’ mainly refer to salts and di-, oligo- and/or polysaccharides.

| | |
|---|---|
| Arabinose | 41.7 |
| Galactose | 1.8 |
| Rhamnose | 0.8 |
| Xylose | 1.4 |
| Others | 54.2 |

### EXAMPLE 13

Enzymatic Hydrolysis of Gum Arabic

4 g of gum arabic (Gum Seyal, Valmar, Valspray, France) was solubilized in 16 ml of water to obtain a 20% gum arabic solution. The temperature of the solution was adjusted to 40° C. and the pH of the solution was adjusted to 5.0 with 1M NaOH solution. To start enzyme hydrolysis, 0.2 ml of an arabinofuranosidase enzyme preparation (having an arabinofuranosidase activity of 58.8 U/g and an arabinanase activity of 3.8 U/g, manufactured by Gist-Brocades) was added. The hydrolysis was continued at 40° C. for 26 hours. To stop the hydrolysis, the solution was heated to 80° C. for 30 minutes to inactivate the enzymes, followed by filtration with a Büchner funnel and paper using diatomaceous earth as a filter aid. The sugar content of the hydrolyzate was determined. The results are presented in the following table (expressed in % on DS). ‘Others’ mainly refer to salts and di-, oligo- and/or polysaccharides.

| | |
|---|---|
| Arabinose | 15.0 |
| Galactose | 0.8 |
| Rhamnose | 2.0 |
| Others | 82.2 |

### EXAMPLE 14

Selective Hydrolysis of the Front-End Fraction Obtained from the Chromatographic Fractionation of Example 2(B)

This example describes the hydrolysis of the front-end fraction containing poly-, oligo- and disaccharides obtained from Example 2(B) (before the arabinose main peak). 59.3 l of water was heated in a reactor to 70° C. and 4.58 kg of 93% H₂SO₄ was added to the reactor. 39 kg of the front-end solution collected from the fractionation of Example 2(B) was concentrated and added to the reactor resulting in a solution having an RDS of 16.9%. The hydrolysis was started by heating the solution to 98° C. in 10 minutes. The hydrolysis was then continued for 5 hours with gentle agitation while keeping the temperature in the range of 97 to 99° C. The hydrolysis was stopped by cooling the solution to 40° C. and neutralizing with 20% Ca(OH)₂ to a pH of 3.12. The solution was filtered with a Seitz filter press and paper filter using diatomaceous earth as a filter aid to remove the insoluble substance. The hydrolyzate thus obtained had an RDS of 9.2%. The sugar content of the hydrolyzate (expressed in % of RDS of the feed solution) is presented in the following table:

| | Rhamnose, | Arabinose, | Galactose, |
|---|---|---|---|
| Time, h | % on RDS | % on RDS | % on RDS |
| 0 | 0.87 | 1.26 | 1.19 |
| 1 | 1.38 | 4.83 | 22.34 |
| 2 | 1.46 | 4.91 | 30.84 |
| 3 | 1.47 | 4.85 | 32.43 |
| 4 | 1.47 | 4.89 | 33.74 |
| 5 | 1.48 | 4.89 | 34.54 |
| After neutralization | 1.75 | 5.78 | 40.84 |
| After filtration | 1.67 | 5.64 | 39.93 |

### EXAMPLE 15

Crystallization of Arabinose from Arabinose-Containing Solutions with Different Galactose Contents

(A) Cooling Crystallization of Arabinose

Cooling crystallization of arabinose was carried out from arabinose fraction obtained from three different chromatographic fractionations of a gum arabic hydrolyzate. Furthermore, crystallization of arabinose was carried out from mother liquors obtained from said three crystallizations. A total of six crystallization tests were thus made in the same crystallization conditions to demonstrate the critical effect of the galactose content on the quality of the arabinose crystals. The solutions used for the crystallization tests were the following:
Test 1: Arabinose fraction obtained from the chromatographic fractionation with a resin in Na⁺ form (fractionation in accordance with Example 3)
Test 2: Arabinose fraction obtained from the chromatographic fractionation with a resin in Ca²⁺ form (fractionation in accordance with example 2B)
Test 3: Arabinose fraction obtained from the chromatographic fractionation with a resin in Ca²⁺ form (fractionation in accordance with example 2B) followed by cation and anion exchange
Test 4: Mother liquor from the crystallization of test 1
Test 5: Mother liquor from the crystallization of test 2
Test 6: Mother liquor from the crystallization of test 3.

The cooling crystallization was carried out as follows:

The feed solution was evaporated at a low pressure to the seeding RDS and the syrup thus obtained was transferred into a 6-liter cooling crystallizer at 60° C. The syrup was seeded at 60° C. with dry arabinose seed crystals in an amount of 0.06% of the DS of the syrup. After seeding, the mass was cooled from 60° C. to 25° C. in 35 hours. The crystallization mass was subjected to centrifuging tests with a laboratory basket centrifuge Roto Silenta 11 (15 min with 3500 rpm) using 0, 100 and 200 ml of washing water. The crystal cake samples were dried at 40° C. overnight and analyzed.

(B) Boiling and Cooling Crystallization of Arabinose

Additionally, a further crystallization test (test No. 7) was made by boiling and cooling crystallization from the same feed liquor as in test 1 above to demonstrate the benefits of boiling crystallization. Boiling crystallization was made by evaporating the feed syrup at a reduced pressure. Seeding was made at 63.8° C. at an RDS % of 64.1 by adding 0.01% of dry arabinose seed crystals. After seeding, the boiling crystallization was continued for about 1 hour until the RS of the mass was 65.8% (density 1.306 kg/l). The crystallization mass was then cooled from 65° C. to 25° C. in 35 hours. Thereafter, centrifuging tests were made with a pilot batch centrifuge and the crystal cake samples were collected in the same way as in Item (A) above.

(C) Results of Crystallization Tests 1 to 7

The crystallization parameters and the results of tests 1 to 7 are presented in Table A. Table A shows the melting points (m.p) of arabinose measured by two different methods: m.p (DSC) is measured as a peak temperature by the differential scanning calorimetric method with a heating rate of 10° C./min and m.p. (Eur:Ph) is measured with the European Pharmacopeia method.

The productivity of the crystallization method during the cooling phase is expressed as the amount of produced crystals (kg) per crystal mass volume (m³) per cooling time (h).

TABLE A
Crystallization parametres and the results of the arabinose crystallization tests

| Test: | | | | | | |
|---|---|---|---|---|---|---|
| 1 | 2 | 3 | 4 | 5 | 6 | 7 |

Crystallization parameters

| | | | | | | | |
|---|---|---|---|---|---|---|---|
| Feed liquor: | | | | | | | |
| RDS, % | 36.3 | 36.7 | 41.3 | 45.3 | 44.1 | 46.4 | 36.3 |
| Arabinose, % on RDS | 91.5 | 88.1 | 88.1 | 77.2 | 77.5 | 79.4 | 90.2 |
| Galactose, % on RDS | 2.1 | 1.6 | 1.6 | 4.7 | 2.5 | 3.3 | 2.1 |
| PH | 5.8 | 3.1 | 3.2 | 5.1 | 3.1 | 3.2 | 5.6 |
| Color, Icumsa | 2700 | 850 | 50 | 4500 | 1600 | 130 | 2500 |
| Seeding: | | | | | | | |
| RDS, % | 65.6 | 63.6 | 64.5 | 66.2 | 64.2 | 64.3 | 64.1 |
| Temperature, ° C. | 60 | 59 | 59.1 | 60.6 | 59.9 | 60 | 63.8 |
| Seeds, % on DS | 0.07 | 0 | 0.07 | 0.07 | 0.07 | 0.07 | 0.01 |
| Seeds, g | 3.2 | 0 | 5.2 | 3.9 | 2.6 | 2.7 | 20 |
| Cooling start, ° C. | 60 | 59 | 59.5 | 60 | 60 | 60 | 65 |
| Cooling end, ° C. | 25 | 25 | 25 | 25 | 25 | 25 | 25 |
| Cooling time, h | 35 | 35 | 35 | 35 | 35 | 35 | 35 |

Crystallization results

| | | | | | | | |
|---|---|---|---|---|---|---|---|
| Wash water, ml | 0 | 0 | 0 | 0 | 0 | 0 | 0 |
| Yield, % on arabinose | 64.8 | 54.4 | 52.7 | 44.5 | 45.8 | 48 | 69.1 |
| Color, ICUMSA | 300 | 95 | 7 | 660 | 200 | 23 | 270 |
| Purity, % arabinose | 99.1 | 98.7 | 98.7 | 98.2 | 99.3 | 98.1 | 98.8 |
| Galactose, % on RDS | 0.4 | 0.5 | 0.4 | 1.1 | 0.5 | 0.5 | 0.5 |
| M.p.(DSC), ° C. | 163 | 163.3 | 165.2 | 158.4 | 159.1 | 162.5 | 161.5 |
| M.p. (Eur. Ph.), ° C. | 157.7 | | | 155.4 | | 156.3 | |
| Productivity, kg/m3/h | 14.5 | 11.3 | 11.1 | 8.5 | 8.4 | 9.1 | 15.3 |
| Wash water, ml | 100 | 100 | 100 | 100 | 100 | 100 | 6 sec |
| Yield, % on arabinose | 62.5 | 49.7 | 48.2 | 45 | 41.4 | 44.3 | 64.3 |
| Color, ICUMSA | 180 | 23 | 1 | 270 | 60 | 5 | 100 |
| Purity, % arabinose | 99.2 | 99.7 | 99.7 | 99 | 99.3 | 99.3 | 99.5 |
| Galactose, % on RDS | 0.3 | 0.3 | 0.2 | 0.8 | 0.5 | 0.5 | 0.3 |
| M.p. (DSC), ° C. | 164.5 | 165 | 164.5 | 162.8 | 164.6 | 164 | 165.1 |
| M.p. (Eur. Ph.), ° C. | 157.5 | | | | | | |
| Wash water, ml | 200 | 200 | 200 | 200 | 200 | 200 | 15 sec |
| Yield, % on arabinose | 59 | 47.9 | 45.3 | 40.6 | 37.5 | 41.7 | 58.5 |
| Color, ICUMSA | 140 | 5 | 2 | 165 | 32 | 10 | 51 |
| Purity, % arabinose | 99.2 | 99.7 | 99.8 | 99.3 | 99.6 | 99.4 | 99.7 |
| Galactose, % on RDS | 0.4 | 0.3 | 0.2 | 0.7 | 0.4 | 0.4 | 0.3 |
| M.p.(DSC), ° C. | 165.9 | 165.4 | 165.3 | 164.1 | 163.3 | 163.9 | 166 |
| M.p.(Eur. Ph.), ° C. | 158.9 | 158.5 | 158.7 | 157.2 | 157.2 | 157.3 | 158.6 |

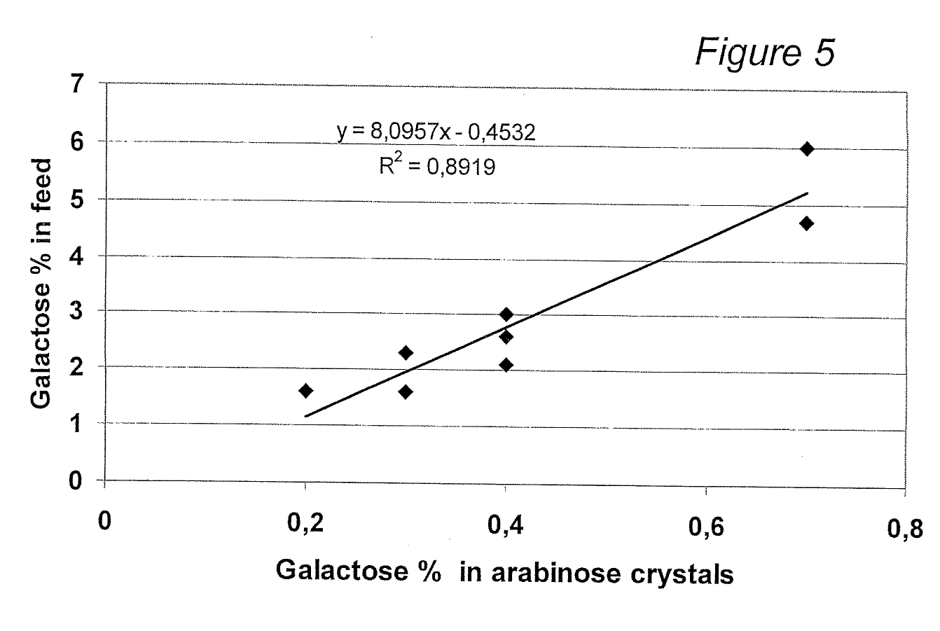

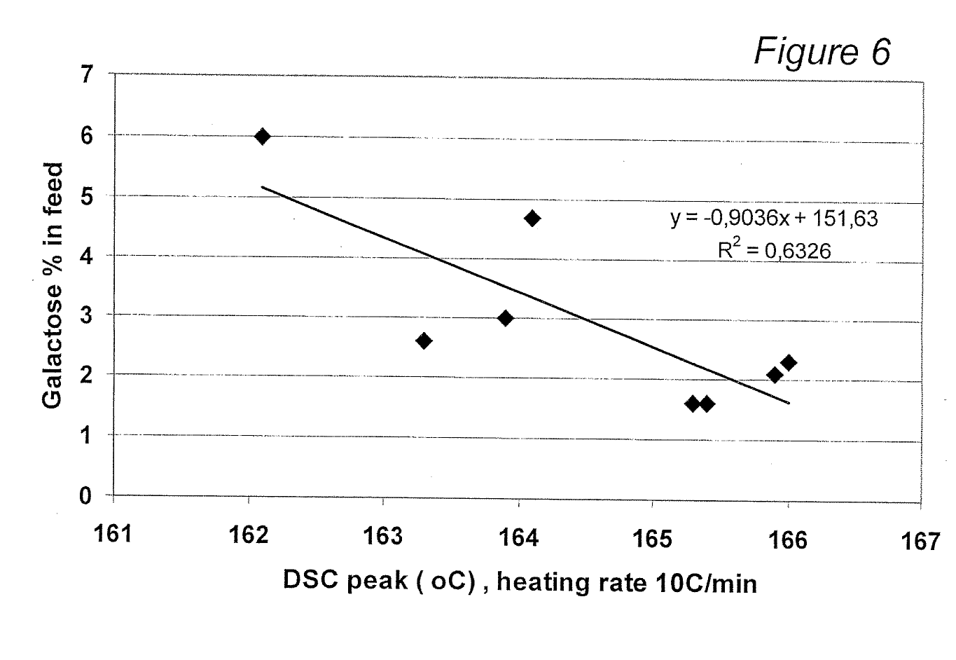

Furthermore, FIG. 5 shows the effect of the galactose content of the crystallization feed on the purity of the arabinose crystals (the content of arabinose in the crystals). FIG. 6 shows the effect of the galactose content of the crystallization feed on the melting point of the arabinose crystals.

The results demonstrate that there is a strong correlation between the galactose content of the feed syrup and the crystal properties after excess washing. Said correlation factor between the galactose content of the crystals and the galactose content of the feed liquor has a value of 0.89. On the contrary, there is no correlation between the purity (the arabinose content) of the feed syrup) and the crystal properties after excess washing (said correlation factor between the galactose content of the crystals and the purity of the feed liquor has a value of only 0.11).

Crystals containing less than 0.5% galactose can be obtained only if there is less than 5% galactose in the feed syryp. Crystals containing less than 0.2% galactose can be prepared only if there is less than 2% galactose in the feed syrup.

The results of Table A also show that, boiling and cooling crystallization has turned out to be the preferred method for crystallizing arabinose from an aqueous solution. The crystal quality, for example the crystal size distribution of arabinose, was improved during boiling crystallization, which can be seen from the better results of the centrifuging tests (yield, color removal, crystal purity) as compared to those of the cooling crystallization tests.

(D) Comparison to Commercial Arabinose

Furthermore, commercial samples of crystalline L-arabinose were analyzed as reference samples. The analyzed commercial arabinose products were Merck 27 (=Merck 1491 lot 6415027), Merck01 (=Merck 1492 lot 0092301), Merck57 (=Merck 1492 lot 31060557) and Merck22 (=Merck 1.01492 lot K21356492622) (all manufactured by Merck), Aros99 (=99+%, lot A0124A012464901) (manufactured by Acros Organics) and Zoster35.048 (manufactured by Zoster). The results of the analysis are shown in Table B.

TABLE B
Analysis of commercial L-arabinose crystals

| Commercial crystal | Merck27 | Merck01 | Merck57 | Merck22 | Aros99 | Zoster35.048 |
|---|---|---|---|---|---|---|
| Purity, % arabinose | 99.9 | 99.7 | 99.9 | 99.5 | 99.7 | 97.9 |
| Galactose, % on RDS | 0.1 | 0.2 | 0.1 | 0.2 | 0.2 | 0.6 |
| M.p (DSC), ° C. | 162.4 | 159.9 | 162.6 | 159.4 | 163.4 | 158.5 |
| M.p (Eur. Ph.), ° C. | 157.5 | 156.2 | 157.2 | 157.1 | 156.8 | 154.2-163.8 |

It appears from the results that commercial arabinose crystals show a melting point which is about 3 to 5° C. lower than that of the arabinose crystals of the present invention when measured by DSC. As a general conclusion it can be stated that the melting point of the arabinose product obtained by the present invention is higher than or at least as high as that of the standard arabinose preparations available on the market. It is thus apparent that the present invention provides one effective way of obtaining very pure crystalline arabinose, which is useful for pharmaceutical or food applications, for example. In accordance with the present invention, arabinose crystals having a high purity and a low galactose content are thus advantageously obtained by single-stage crystallization without dissolving and recrystallization steps.

It will be obvious to a person skilled in the art that as technology advances, the inventive concept can be implemented in various ways. The invention and its embodiments are not limited to the examples described above but may vary within the scope of the claims.

1. A process of recovering arabinose and optionally at least one other monosaccharide selected from the group consisting of galactose, rhamnose and mannose from vegetable fiber rich in heteropolymeric arabinose, wherein the process comprises the following steps:
(a) controlled acidic or enzymatic hydrolysis of said vegetable fiber in an aqueous solution to hydrolyze more than 50% of the heteropolymeric arabinose present in the vegetable fiber into monomeric arabinose and to pro- duce an aqueous hydrolyzate containing at least 10% arabinose on DS, at least one other monosaccharide selected from the group consisting of galactose and optionally rhamnose and mannose, and optionally poly-, oligo- and/or disaccharides, soluble polymers and undissolved solids,
(b) optional neutralization of said aqueous hydrolyzate to obtain a neutralized hydrolyzate,
(c) separation of undissolved solids from said aqueous hydrolyzate obtained in step (a) or from said neutralized hydrolyzate obtained in step (b) to obtain a clarified hydrolyzate,
(d) optional fractionation of said clarified hydrolyzate in an aqueous solution to obtain a fraction enriched in arabinose, which contains at least 50% arabinose and less than 30% of one or more monosaccharides selected from galactose and optionally rhamnose and mannose on DS, at least one other fraction selected from the group consisting of a fraction enriched in galactose, a fraction enriched in rhamnose and a fraction enriched in mannose, and optionally one or more fractions enriched in poly-, oligo- and/or disaccharides and soluble polymers, followed by the recovery of said fraction enriched in arabinose and optionally one or more of said other fractions, and
(e) crystallization of arabinose in an aqueous solution from said hydrolyzate obtained in step (c) or from said fraction enriched in arabinose obtained in step (d) to obtain crystalline arabinose having a galactose content of less than 1% on DS.

2. A process as claimed in claim 1, wherein said vegetable fiber rich in heteropolymeric arabinose contains more than 15% arabinose on DS.

3. A process as claimed in claim 2, wherein said vegetable fiber contains more than 35% arabinose on DS.

4. A process as claimed in claim 2, wherein said vegetable fiber rich in heteropolymeric arabinose is an exudate gum.

5. A process as claimed in claim 4, wherein said exudate gum is selected from gum arabic, gum ghatti and gum tragacanth.

6. A process as claimed in claim 1, wherein said vegetable fiber rich in heteropolymeric arabinose is sugar beet pulp.

7. A process as claimed in claim 1, wherein said vegetable fiber rich in heteropolymeric arabinose is selected from hardwood bark, grain straw and hulls, corn husks, corn cobs, corn fibers and bagasse.

8. A process as claimed in claim 7, wherein said hardwood bark is selected from beech bark and birch bark.

9. A process as claimed in claim 1, wherein said vegetable fiber rich in heteropolymeric arabinose is water-soluble or alkali soluble vegetable fiber.

10. A process as claimed in claim 1, wherein said controlled hydrolysis of step (a) provides an aqueous hydrolyzate where more than 70% of said heteropolymeric arabinose is hydrolyzed into monomeric arabinose.

11. A process as claimed in claim 10, wherein more than 80% of said heteropolymeric arabinose is hydrolyzed into monomeric arabinose.

12. A process as claimed in claim 1, wherein said controlled hydrolysis of step (a) provides an aqueous hydrolyzate where the content of arabinose is more than 15% on DS.

13. A process as claimed in claim 12, wherein the content of arabinose is more than 20% on DS.

14. A process as claimed in claim 1, wherein said hydrolysis is carried out as a selective hydrolysis by adjusting the hydrolysis conditions so as to obtain a hydrolyzate where the content of galactose is less than 10% on DS.

15. A process as claimed in claim 14, wherein the content of galactose is less than 5% on DS.

16. A process as claimed in claim 15, wherein the content of galactose is less than 2% on DS.

17. A process as claimed in claim 1, wherein said hydrolysis is carried out in the acid concentration of 0.1-5% and with an acid selected from mineral acids and organic acids.

18. A process as claimed in claim 17, wherein said inorganic acid is sulphuric acid.

19. A process as claimed in claim 17, wherein said hydrolysis is carried out at a temperature in the range of 70 to 140° C., at a pH in the range of 0.7 to 2.5 and the hydrolysis is continued for 0.4 to 6 hours.

20. A process as claimed in claim 1, wherein said fractionation of step (d) is carried out by chromatographic fractionation to obtain a fraction enriched in arabinose, at least one other fraction selected from a fraction enriched in galactose, a fraction enriched in rhamnose and a fraction enriched in mannose, and optionally one or more fractions enriched in poly-, oligo- and/or disaccharides and soluble polymers.

21. A process as claimed in claim 20, wherein said chromatographic fractionation is carried out using a column packing material selected from cation exchange resins.

22. A process as claimed in claim 21, wherein said cation exchange resins are selected from strongly acid cation exchange resins.

23. A process as claimed in claim 22, wherein the ion form of said strongly acid cation exchange resin is selected from H⁺, Na⁺, K⁺, Ca²⁺, Mg²⁺, NH₄⁺Al³⁺, Sr³⁺ and Ba²⁺.

24. A process as claimed in claim 21, wherein said cation exchange resins are selected from weakly acid cation exchange resins.

25. A process as claimed in claim 24, wherein the ion form of said weakly acid cation exchange resins is selected from H⁺, Na⁺ and Ca²⁺.

26. A process as claimed in claim 20, wherein said chromatographic fractionation is carried out using a column packing material selected from anion exchange resins.

27. A process as claimed in claim 26, wherein said anion exchange resins are selected from weakly basic anion exchange resins.

28. A process as claimed in claim 26, wherein said anion exchange resins are selected from strongly basic anion exchange resins.

29. A process as claimed in claim 28, wherein the ion form of said strongly basic anion exchange resin is selected from HS0₃⁻ and S0₄²⁻.

30. A process as claimed in claim 1, wherein said fractionation of step (d) is carried out by membrane filtration.

31. A process as claimed in claim 30, wherein said membrane filtration is carried out bynanofiltration to obtain a fraction enriched in arabinose as the nanofiltration permeate and a fraction enriched in poly-, oligo- and/or disaccharides as the nanofiltration retentate.

32. A process as claimed in claim 1, wherein the process comprises at least two fractionations selected from chromatographic fractionation and/or membrane filtration.

33. A process as claimed in claim 1, wherein the arabinose yield in the fraction enriched in arabinose obtained from step (d) is more than 50% of the arabinose present in the hydrolyzate.

34. A process as claimed in claim 1, wherein said fraction enriched in poly-. oligo- and/or disaccharides is further subjected to hydrolysis to obtain a hydrolyzate containing galactose and optionally rhamnose, mannose and additional arabinose.

35. A process as claimed in claim 34, wherein the process further comprises separating galactose and optionally rhamnose, mannose and additional arabinose from said hydrolyzate obtained in step (c).

36. A process as claimed in claim 1, which further comprises the recovery of a fraction enriched in soluble polymers obtained in step (d).

37. A process as claimed in claim 36, wherein the soluble polymers comprise pectin.

38. A process as claimed in claim 1, which further comprises the separation of xylose from said hydrolyzate as a prefractionation step before the fractionation step (d).

39. A process as claimed in claim 1, wherein said crystallization of arabinose in step (e) is carried out from said hydrolyzate obtained in step (c).

40. A process as claimed in claim 1, wherein said crystallization of arabinose in step (e) is carried out from said fraction enriched in arabinose obtained in step (d).

41. A process as claimed in claim 1, wherein said crystallization of arabinose in step (e) comprises boiling crystallization.

42. A process as claimed in claim 41, wherein said boiling crystallization is combined with cooling crystallization.

43. A process as claimed in claim 1, wherein said crystallization of arabinose comprises cooling crystallization.

44. A process as claimed in claim 1, wherein said crystallization of arabinose in step (e) is carried out from a solution having an arabinose purity of more than 60% on DS.

45. A process as claimed in claim 44, wherein the arabinose purity is more than 70% on DS.

46. A process as claimed in claim 1, wherein said crystallization of arabinose in step (e) is carried out in the presence of less than 10% galactose on DS as an impurity.

47. A process as claimed in claim 46, wherein said crystallization of arabinose is carried out in the presence of less than 5% galactose on DS as an impurity.

48. A process as claimed in claim 47, wherein said crystallization of arabinose is carried out in the presence of less than 2% galactose as an impurity.

49. A process as claimed in claim 1, wherein said crystallization of arabinose in step (e) comprises a single-stage crystallization.

50. A process as claimed in claim 1, wherein said crystallization of arabinose in step (e) further comprises washing the crystals obtained from the crystallization.

51. A process as claimed in claim 1, wherein said crystallization of arabinose in step (e) provides a crystalline arabinose product having a purity of more than 60% on DS.

52. A process as claimed in claim 51, wherein the purity of the arabinose product is more than 70% on DS.

53. A process as claimed in claim 52, wherein said crystallization provides crystalline arabinose having a purity of more than 90% on DS.

54. A process as claimed in claim 53, wherein the purity of the crystalline arabinose is more than 98% on DS.

55. A process as claimed in claim 54, wherein the purity of the crystalline arabinose is more than 99% on DS.

56. A process as claimed in claim 55, wherein the purity of crystalline arabinose is more than 99.5% on DS.

57. A process as claimed in claim 1, wherein the arabinose yield in the crystallization in step (e) is more than 40%.

58. A process as claimed in claim 57, wherein the arabinose yield is more than 60%.

59. A process as claimed in claim 58, wherein the arabinose yield is more than 80%.

60. A process as claimed in claim 1, wherein the crystalline arabinose obtained in step (e) has a galactose content of less than 0.5% on DS.

61. A process as claimed in claim 60, wherein the galactose content is less than 0.2% on DS.

62. A process for the crystallization of arabinose from a biomass-derived solution, wherein said crystallization comprises a single-stage boiling crystallization in an aqueous solution from a biomass derived solution having an arabinose purity of more than 70% on DS.

63. A process as claimed in claim 62, wherein said crystallization is carried out in the presence of less than 10% galactose on DS as an impurity.

64. A process as claimed in claim 63, wherein said crystallization is carried out in the presence of less than 5% galactose on DS as an impurity.

65. A process as claimed in claim 64, wherein said crystallization is carried out in the presence of less than 2% galactose on DS as an impurity.

66. A process as claimed in claim 62, wherein said crystallization further comprises washing of the arabinose crystals.

67. A process as claimed in claim 66, wherein said crystallization of arabinose provides crystalline arabinose having a purity of more than 98% on DS.

68. A process as claimed in claim 67, wherein the purity of crystalline arabinose is more than 99% on DS.

69. A process as claimed in claim 68, wherein the purity of crystalline arabinose is more than 99.5% on DS.

70. A process as claimed in claim 1, wherein the process comprises a further step of subjecting crystallized arabinose or said fraction enriched in arabinose to epimerization to convert arabinose to ribose.

71. A process as claimed in claim 1, wherein said arabinose is L-arabinose.

72. Crystalline L-arabinose based on vegetable fiber, which has a melting point higher than 164° C. determined by DSC with a heating rate of 10° C./min, a melting point higher than 158° C. determined by the European Pharmacopeia method and a purity of more than 99.5% on DS.

73. Crystalline L-arabinose as claimed in claim 72, which has a melting point higher than 165° C. determined by DSC with a heating rate of 10° C./min.

74. Crystalline L-arabinose as claimed in claim 72, which contains galactose in an amount of less than 0.5% on DS.

75. Crystalline L-arabinose as claimed in claim 73, which contains galactose in an amount of less than 0.2% on DS.

76. Crystalline L-arabinose as claimed in claim 72, which is obtainable by boiling crystallization of arabinose.

77. Crystalline L-arabinose as claimed in claim 76, which is obtainable by combined boiling and cooling crystallization of arabinose.

78. (canceled)

79. (canceled)

80. A process as claimed in claim 62, wherein the process comprises a further step of subjecting crystallized arabinose or said fraction enriched in arabinose to epimerization to convert arabinose to ribose.

81. A process as claimed in claim 62, wherein said arabinose is L-arabinose.
